(19) United States
(12) Patent Application Publication
(10) Pub. No.: US 2008/0070984 A1
(43) Pub. Date: Mar. 20, 2008

(54) Compositions and Methods of Treating Schizophrenia

(76) Inventor: Pierre V. TRAN, San Jose, CA (US)

Correspondence Address: Timothy A. Worrall;Dorsey & Whitney LLP, Intellectual Property Department, 555 California Street, 3rd Floor, San Francisco, CA, 94104-1513, US

(21) Appl. No.: 11/855,641

(22) Filed: Sep. 14, 2007

Related U.S. Application Data
(60) Provisional application No. 60/845,225, filed on Sep. 15, 2006.

(51) Int. Cl. A61K 31/195; A61K 31/225; A61K 31/235; A61P 25/18

(52) U.S. Cl. 514/533; 514/538; 514/567

(57) ABSTRACT

Compositions and methods of treating both the positive and negative or cognitive symptoms of schizophrenia are disclosed. More specifically, pharmaceutical compositions for oral administration comprising at least one antipsychotic agent in an amount that is effective for treating a positive symptom of schizophrenia and at least one colonically absorbable form of levodopa in an amount that is effective for treating a negative or cognitive symptom of schizophrenia and the use of such compositions for treating schizophrenia are disclosed.

This application claims benefit of U.S. Provisional Application No. 60/845,225 filed Sep. 15, 2006, which is incorporated by reference herein in its entirety.

### FIELD

The disclosure herein relates generally to compositions and methods of treating schizophrenia.

### BACKGROUND

Schizophrenia is a chronic, severe, and disabling brain disorder that affects about one percent of people worldwide, including 3.2 million Americans. Schizophrenia is among the top ten causes of disability in the United States, accounting for 20% of Social Security disability days (Williams and Dickson, Can J Psychiatry 1995, 40(7, Suppl. 2), S60-S67). The resulting economic costs of schizophrenia are estimated to be $65 billion per year with direct costs related to patient and out-patient care of $19 billion and indirect costs attributed to lost productivity and lost income of $46.5 billion (Wyatt et al., Soc Psychiatr Epidemiol 1995, 30, 196-205).

Because the causes of schizophrenia remain largely unknown, current treatments focus on controlling the symptoms of the disease. The symptoms of schizophrenia fall into three broad categories. Positive symptoms are unusual thoughts or perceptions that include hallucinations, delusions, thought disorder, excessive behavior, distorted perceptions of reality, disorganized speech, and disorganized or otherwise bizarre behavior. Negative symptoms refer to reductions in normal emotional and behavioral states. These include flat affect (immobile facial expression, monotonous voice), lack of pleasure in everyday life, social withdrawal, diminished ability to initiate and sustain planned activity, avoidance of eye contact, apathy, and speaking infrequently, even when forced to interact. Cognitive symptoms are subtle and are often detected only when neuropsychological tests are performed. They include poor executive functioning (the ability to absorb and interpret information and make decisions based on that information), inability to sustain attention, and problems with working memory (the ability to keep recently learned information in mind and use it effectively).

Antipsychotic medications have been available to treat schizophrenia since the mid-1950s, and while these drugs have greatly improved the lives of many patients by effectively alleviating the positive symptoms, the drugs are generally not effective for treating the negative and cognitive symptoms of schizophrenia.

The first generation of antipsychotic medications, also referred to as typical antipsychotic drugs, include, for example, chlorporomazine, emonopride, fluphenazine, haloperidol, loxapine, mesoridazine, molindone, pimozide, perphenazine, raclopride, remoxipride, spiperone, thioridazine, thiothixene, and trifluoperazine. Typical antipsychotics are believed to act by blocking dopaminergic receptors (dopamine receptor antagonists), primarily dopamine D2 receptors, thereby reducing dopaminergic transmission in the brain. Unfortunately, these drugs can cause extrapyramidal side (EPS) effects, such as rigidity, persistent muscle spasms, tremors, restlessness, and tardive dyskinesia.

In the 1990s, a new class of antipsychotic drugs, referred to as atypical antipsychotics, was developed that produced these side effects with less frequency. Atypical antipsychotics exhibit a different and recognizable clinical and pharmacological profile relative to typical antipsychotics and exhibit advantages over the typical antipsychotics. Typical antipsychotics, such as haloperidol, are selective antagonists of dopamine D2 receptors. Atypical antipsychotics also have D2 receptor antagonist properties, however their binding kinetics to D2 receptors are different and the antagonist activity to D2 receptors are relatively weak. Furthermore, atypical antipsychotics can act as agonists and/or antagonists at other receptors, such as 5-HT_{2A}, 5-HT_{2c}, and 5-HT_{1d} serotonin receptors. A feature of atypical antipsychotics is reduced EPS effects, especially dystonias, as compared to typical antipsychotics. For example, atypical antipsychotics exhibit greater efficacy in the treatment of overall psychotherapy in schizophrenics nonresponsive to typical antipsychotics, greater efficacy in the treatment of negative symptoms of schizophrenia, less frequent and quantitatively smaller increase in serum prolactin concentrations associated with therapy, lower risks of EPS or tardive dyskinesia, and improved cognitive functions (see, e.g., Beasley, et al., Neuropsychopharmacology 1996, 14(2), 111). Examples of atypical antipsychotics include, but are not limited to, asenapine olanzapine, clozapine, risperidone, sertindole, quetiapine, aripiprazole, amisulpride, ziprasidone, and mirtazapine. Although atypical antipsychotics can be effective in treating the positive symptoms of schizophrenia and produce fewer EPS effects, atypical antipsychotics can also cause weight gain, which may increase the risk of diabetes and high cholesterol, together called metabolic syndrome, and in the case of clozapine, may produce serious side effects such as agranulocytosis, a loss of the white blood cells that fight infection. While in some respects atypical antipsychotics represent a significant improvement to typical antipsychotics, cognitive and psychological deficits continue to persist in many schizophrenia patients treated with atypical antipsychotic drugs (Chakos et al., Am J Psychiatry 2001, 158, 518-526).

Nevertheless, atypical antipsychotic drugs such as clozapine show some efficacy relative to typical antipsychotic drugs in treating schizophrenia not responsive to conventional antipsychotics, and exhibit an improved effect on both the negative symptoms and some aspects of cognitive impairment (Kane, Psychopharmacol Bull, 1988, 24, 62-67, Kane et al., Arch Gen Psychiatry 2001, 58, 965-72; Meltzer, Schizophr Bull 1992, 18, 515-42; and Meltzer and McGurk, Schizophr Bull, 1999, 25, 233-55). Studies show that clozapine and other atypical antipsychotic drugs, such as aripiprazole, in contrast to typical antipsychotic drugs, cause a preferential increase in dopamine in the prefrontal cortex (Imperato and Angelucci, Psychopharmacol Bull 1989, 25, 383-89; Li et al., Eur J Pharmacol, 2004, 493, 75-83; Moghaddan and Bunney, J. Neurochem 1990, 54, 1755-1760; Nomikos et al, Psychopharmacology 1994, 115, 147-156; and Westerink et al., Eur J Pharmacol, 2001, 412, 127-138). The enhanced dopamine efflux in the prefrontal cortex is believed to be responsible for the improvements in the negative and cognitive symptoms of schizophrenia when these drugs are administered, mediated by stimulation of D1 receptors (Castner et al., Science 2000, 287, 2020-2022) and secondarily by increased glutamatergic transmission in the prefrontal cortex (Chen and Yang, J Neurophysiol, 2002, 87(5), 2324-2336; and Ninan and Wang, Eur J Neurosci, 2003, 17, 1306-1312). The enhanced prefrontal dopamine efflux obtained by atypical antipsychotic drugs is generally thought to contribute to their advantageous effects on negative and cognitive symptoms in schizophrenia (see, Svensson, Clin Neurosci Res, 2003, 3, 34-46). Therefore, an analogous effect of adjunctive levodopa treatment when added to selective dopamine D2 antagonists, e.g., typical antipsychotics, should serve a similar function, in particular because clinical results demonstrate an enhanced conversion of levodopa to dopamine in the prefrontal cortex in schizophrenia (Lindstrom et al., Biol Psychiatry 1999, 46, 681-88).

Administration of high doses of a dopamine D2 receptor agonist or precursor thereof such as levodopa, either alone or concomitant with an antipsychotic drug, has been shown to exacerbate psychosis, or even induce psychosis in non-psychotic patients (Lehrman and Sharar, J. Ment Health Admin, 1997, 24, 227-250; Angrist et al, 1973; Yaryura-Tobias et al., Curr Ther Res Clin Exp 1970, 12, 528-31; and Yaryura-Tobias et al., Dis Nerv Syst 1970, 31, 60-63). However, relatively low doses of levodopa given as adjunctive treatment with typical antipsychotic drugs improves the clinical outcome in schizophrenia (see Jaskiw and Popli, Psychopharmacology 2004, 171, 365-374), suggesting an enhanced effect on negative symptoms and cognitive impairment without worsening of psychotic symptoms (Alpert and Friedhoff, Am J Psychiatry 1980, 135, 1329-32; Bruno and Bruno, Acta Psychiatr Scand, 1966, 42, 264-71; Buchanan et al., Aust N Z J Psychiatry 1975, 9, 269-71; Gerlach and Luhdorf, Psychopharmacologia 1975, 44, 105-110; Inanaga et al., Folia Psychiatr Neurol Jpn 1975, 29, 123-43; and Kay and Opler, Int J Psychiat Med 1985-86, 15, 293-98). The results of these studies suggest that adjunctive low-dose levodopa together with a low dose of a conventional antipsychotic drug can be expected to generate a therapeutic profile similar to that of atypical antipsychotic drugs, including enhanced treatment efficacy against negative symptoms and cognitive impairment in schizophrenia, with retained therapeutic effects on positive symptoms and without concomitant increased EPS liability. Because the severity of cognitive impairment has a crucial impact on treatment outcome (Green, Am J Psychiatry 1996, 153, 321-330; and Harvey et al., Am J Psychiatry 1998, 155, 1080-1086) the use of adjunctive, low-dose levodopa with selective dopamine D2 antagonists may also prove efficacious in treating both the positive and negative or cognitive symptoms of schizophrenia.

Unfortunately, levodopa has a short time-to-peak plasma level, a half-life of about 1.5 hours, and poor oral bioavailability, and therefore must be administered at least 3 times per day, which can make it impractical to use in the treatment of mental/psychotic disorders such as schizophrenia. In addition, levodopa is rapidly metabolized to dopamine by L-aromatic amino acid decarboxylase (AADC) enzymes in the peripheral tissues (e.g., intestines and liver). For these reasons, levodopa must be co-administered with a decarboxylase enzyme inhibitor such as carbidopa or benserazide. When administered with carbidopa, the plasma concentration of intact levodopa increases and thus more levodopa becomes available to be transported into the central nervous system where it is converted to dopamine, the active metabolite of levodopa.

Levodopa prodrugs designed to be better absorbed throughout the entire gastrointestinal tract (i.e., from both the small and large intestines) and that exhibit reduced first pass metabolism, and the use of such levodopa prodrugs for treating schizophrenia, are described by Xiang et al., U.S. Application Publication Nos. 2006/0020028 and 2005/0282891, each of which is incorporated by reference herein in its entirety. These levodopa prodrugs can achieve an oral bioavailability of levodopa that is at least two times greater than the oral bioavailability of levodopa when orally administered on an equivalent molar basis. Xiang et al., International Publication No. WO 2007/067495, and U.S. Provisional Application Nos. 60/876,148 and 60/876,144 filed Dec. 21, 2006, each of which is incorporated by reference herein in its entirety, also discloses amorphous and crystalline forms of the compound (2R)-2-phenylcarbonyloxypropyl (2S)-2-amino-3-(3,4-dihydroxyphenyl)propanoate mesylate, and the use of such forms for treating schizophrenia. The levodopa prodrugs described by Xiang et al. can be efficaciously incorporated into oral sustained release formulations to provide sustained systemic exposure to levodopa upon oral administration to a patient.

### SUMMARY

In a first aspect, pharmaceutical compositions comprising at least one antipsychotic agent and at least one colonically absorbable form of levodopa for oral administration are provided.

In a second aspect, methods are provided of treating schizophrenia in a patient comprising orally administering to a patient in need of such treatment at least one antipsychotic agent in an amount that is effective for treating a positive symptom of schizophrenia in the patient, and at least one colonically absorbable form of levodopa in an amount that is effective for treating a negative or cognitive symptom of schizophrenia in the patient and that does not exacerbate or induce a positive symptom of schizophrenia in the patient.

In a third aspect, methods are provided of treating schizophrenia in a patient comprising administering to a patient in need of such treatment at least one antipsychotic agent in an amount that is effective for treating a positive symptom of schizophrenia in the patient, and orally administering to the patient, at least one colonically absorbable form of levodopa in an amount that is effective for treating a negative or cognitive symptom of schizophrenia in the patient and that does not exacerbate or induce a positive symptom of schizophrenia in the patient.

### DETAILED DESCRIPTION

### Definitions

A dash (“-”) that is not between two letters or symbols is used to indicate a point of attachment for a substituent. For example, —CONH₂ is a moiety bonded through the carbon atom.

“Alkyl,” by itself or as part of another substituent, refers to a saturated or unsaturated, branched, or straight-chain, monovalent hydrocarbon radical derived by the removal of one hydrogen atom from a single carbon atom of a parent alkane, alkene, or alkyne. Examples of alkyl groups include, but are not limited to, methyl; ethyls such as ethanyl, ethenyl, and ethynyl; propyls such as propan-1-yl, propan-2-yl, prop-1-en-1-yl, prop-1-en-2-yl, prop-2-en-1-yl (allyl); prop-1-yn-1-yl, prop-2-yn-1-yl, etc.; butyls such as butan-1-yl, butan-2-yl, 2-methyl-propan-1-yl, 2-methyl-propan-2-yl, but-1-en-1-yl, but-1-en-2-yl, 2-methyl-prop-1-en-1-yl, but-2-en-1-yl, but-2-en-2-yl, buta-1,3-dien-1-yl, buta-1,3-dien-2-yl, but-1-yn-1-yl, but-1-yn-3-yl, but-3-yn-1-yl, etc.

The term “alkyl” is specifically intended to include groups having any degree or level of saturation, i.e., groups having exclusively single carbon-carbon bonds, groups having one or more double carbon-carbon bonds, groups having one or more triple carbon-carbon bonds, and groups having mixtures of single, double, and triple carbon-carbon bonds. Where a specific level of saturation is intended, the terms “alkanyl,” “alkenyl,” and “alkynyl” are used. In certain embodiments, alkyl groups may comprise from 1 to 20 carbon atoms, in certain embodiments, from 1 to 10 carbon atoms, and in certain embodiments, from 1 to 6 carbon atoms.

“Alkoxy,” by itself or as part of another substituent, refers to the radical —OR⁵¹ where R⁵¹ is alkyl, cycloalkyl, cycloalkylalkyl, aryl, or arylalkyl, which may be substituted, as defined herein. Examples of alkoxy groups include, but are not limited to, methoxy, ethoxy, propoxy, butoxy, cyclohexyloxy, and the like.

“Alkoxycarbonyl,” by itself or as part of another substituent, refers to the radical —C(O)OR⁵² where R⁵² represents an alkyl, as defined herein. Examples of alkoxycarbonyl groups include, but are not limited to, methoxycarbonyl, ethoxycarbonyl, propoxycarbonyl, butoxycarbonyl, and the like.

“Aryl,” by itself or as part of another substituent, refers to a monovalent aromatic hydrocarbon radical derived by the removal of one hydrogen atom from a single carbon atom of a parent aromatic ring system. Aryl encompasses 5- and 6-membered carbocyclic aromatic rings, for example, benzene; bicyclic ring systems wherein at least one ring is carbocyclic and aromatic, for example, naphthalene, indane, and tetralin; and tricyclic ring systems wherein at least one ring is carbocyclic and aromatic, for example, fluorene. Aryl encompasses multiple ring systems having at least one carbocyclic aromatic ring fused to at least one carbocylic aromatic ring, cycloalkyl ring, or heterocycloalkyl ring. For example, aryl includes 5- and 6-membered carbocyclic aromatic rings fused to a 5- to 7-membered heterocycloalkyl ring containing one or more heteroatoms chosen from N, O, and S. For such fused, bicyclic ring systems wherein only one of the rings is a carbocyclic aromatic ring, the point of attachment may be at the carbocyclic aromatic ring or the heterocycloalkyl ring. Examples of aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexylene, s-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like. In certain embodiments, aryl groups have from 5 to 20 carbon atoms, and in certain embodiments, from 5 to 12 carbon atoms.

“Arylalkyl,” by itself or as part of another substituent, refers to an acyclic alkyl radical in which one of the hydrogen atoms bonded to a carbon atom, typically a terminal or sp³ carbon atom, is replaced with an aryl group. Examples of arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. Where specific alkyl moieties are intended, the nomenclature arylalkanyl, arylalkenyl, or arylalkynyl is used. In certain embodiments, an arylalkyl group is C₇₋₃₀ arylalkyl, e.g., the alkanyl, alkenyl, or alkynyl moiety of the arylalkyl group is C₁₋₁₀ and the aryl moiety is C₆₋₂₀, and in certain embodiments, an arylalkyl group is C₇₋₂₀ arylalkyl, e.g., the alkanyl, alkenyl, or alkynyl moiety of the arylalkyl group is C₁₋₈ and the aryl moiety is C₆₋₁₂.

“AUC” is the area under a curve representing the concentration of a compound or metabolite thereof in a biological fluid in a patient as a function of time following administration of the compound to the patient. In certain embodiments, the compound is a prodrug and the metabolite is a drug. Examples of biological fluids include plasma and blood. The AUC may be determined by measuring the concentration of a compound or metabolite thereof in a biological fluid such as the plasma or blood using methods such as liquid chromatography-tandem mass spectrometry (LC/MS/MS), at various time intervals, and calculating the area under the plasma concentration-versus-time curve. Suitable methods for calculating the AUC from a drug concentration-versus-time curve are well known in the art. For example, an AUC for an antipsychotic agent or levodopa may be determined by measuring the concentration of the antipsychotic agent or levodopa in the plasma or blood of a patient following administration of an antipsychotic agent or form of levodopa, respectively, to the patient.

“Bioavailability” refers to the rate and amount of a drug that reaches the systemic circulation of a patient following administration of the drug or prodrug thereof to the patient and can be determined by evaluating, for example, the plasma or blood concentration-versus-time profile for the drug. Parameters useful in characterizing a plasma or blood concentration-versus-time curve include the area under the curve (AUC), the time to peak concentration (Tₘₐₓ), and the maximum drug concentration (Cₘₐₓ).

“Cₘₐₓ” is the maximum concentration of a drug in the plasma or blood of a patient following administration of a dose of the drug or prodrug to the patient.

“Tₘₐₓ” is the time to the maximum concentration (Cₘₐₓ) of a drug in the plasma or blood of a patient following administration of a dose of the drug or prodrug to the patient.

“Compounds of Formulae (I)-(VI)” include any specific compounds within these formulae for which the structure is disclosed herein or incorporated by reference. Compounds may be identified by their chemical structure and/or chemical name. If the chemical structure and chemical name conflict, the chemical structure is determinative of the identity of the compound.

Compounds described herein may comprise one or more chiral centers and/or double bonds and therefore may exist as stereoisomers such as double-bond isomers (i.e., geometric isomers), enantiomers, and diastereomers. Accordingly, any chemical structures within the scope of the specification depicted, in whole or in part, with a relative configuration encompass all possible enantiomers and stereoisomers of the illustrated compounds including the stereoisomerically pure form (e.g., geometrically pure, enantiomerically pure, or diastereomerically pure) and enantiomeric and stereoisomeric mixtures. Enantiomeric and stereoisomeric mixtures may be resolved into their component enantiomers or stereoisomers using separation techniques and stereoisomerically pure forms may be synthesized using chiral synthesis techniques well known to the skilled artisan.

Compounds of Formulae (I)-(VI) include, but are not limited to, optical isomers of compounds of Formulae (I)-(VI), racemates thereof, and other mixtures thereof. In such embodiments, the single enantiomers or diastereomers, i.e., optically active forms, may be obtained by asymmetric synthesis or by resolution of the racemates. Resolution of the racemates may be accomplished, for example, by conventional methods such as crystallization in the presence of a resolving agent, or chromatography, using, for example a chiral high-pressure liquid chromatography (HPLC) column. In addition, compounds of Formulae (I)-(VI) include Z- and E-forms (or cis- and trans-forms) of compounds with double bonds. In embodiments in which compounds of Formulae (I)-(VI) exist in various tautomeric forms, compounds of Formulae (I)-(VI) include any and all tautomeric forms of the compound.

Compounds of Formulae (I)-(VI) may also exist in several tautomeric forms including the enol form, the keto form, and combinations thereof. Accordingly, the chemical structures disclosed herein encompass any and all possible tautomeric forms of the compounds. The compounds of Formulae (I)-(VI) also include isotopically labeled compounds where one or more atoms have an atomic mass different from the atomic mass conventionally found in nature. Examples of isotopes that may be incorporated into the compounds disclosed herein include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated, or N-oxides. Certain compounds may exist in multiple crystalline or amorphous forms.

In general, all physical forms are equivalent for the uses contemplated herein and are intended to be within the scope provided by the present disclosure. Further, it should be understood, when partial structures of the compounds are illustrated, that an asterisk indicates the point of attachment of the partial structure to the rest of the molecule.

“Cycloalkyl,” by itself or as part of another substituent, refers to a partially saturated or unsaturated cyclic alkyl radical. Where a specific level of saturation is intended, the nomenclature “cycloalkanyl” or “cycloalkenyl” is used. Cycloalkyl encompasses multiple ring systems in which none of the rings are aromatic. Examples of cycloalkyl groups include, but are not limited to, groups derived from cyclopropane, cyclobutane, cyclopentane, cyclohexane, and the like. In certain embodiments, a cycloalkyl group is C₃₋₁₅ cycloalkyl, and in certain embodiments, C₅₋₁₂ cycloalkyl.

“Cycloheteroalkyl,” by itself or as part of another substituent, refers to a partially saturated or unsaturated cyclic alkyl radical in which one or more carbon atoms (and any associated hydrogen atoms) are independently replaced with the same or different heteroatom. Cycloheteroalkyl encompasses multiple ring systems in which none of the rings are aromatic and in which at least one ring atom is a heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Examples of cycloheteroalkyl groups include, but are not limited to, groups derived from epoxides, azirines, thiiranes, imidazolidine, morpholine, piperazine, piperidine, pyrazolidine, pyrrolidine, quinuclidine, etc.

“Halogen” refers to a fluoro, chloro, bromo, or iodo group.

“Heteroalkyl,” by itself or as part of another substituent, refer to an alkyl group in which one or more of the carbon atoms (and any associated hydrogen atoms) are independently replaced with the same or different heteroatomic groups. Examples of heteroatomic groups include, but are not limited to, —O—, —S—, —O—O—, —S—S—, —O—S—, —NR⁵³R⁵⁴—, ═N—N═, —N═N—, —N═N—NR⁵⁵R⁵⁶, —PR⁵⁷—, —P(O)₂—, —POR⁵⁸—, —O—P(O)₂—, —SO—, —SO₂—, —SnR⁵⁹R⁶⁰— and the like, where R⁵³, R⁵⁴, R⁵⁵, R⁵⁶, R⁵⁷, R⁵⁸, R⁵⁹, and R⁶⁰ are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl. Where a specific level of saturation is intended, the nomenclature “heteroalkanyl,” “heteroalkenyl,” or “heteroalkynyl” is used.

“Heteroaryl,” by itself or as part of another substituent, refers to a monovalent heteroaromatic radical derived by the removal of one hydrogen atom from a single atom of a parent heteroaromatic ring system. Heteroaryl encompasses multiple ring systems having at least one aromatic ring fused to at least one other ring, which may be aromatic or non-aromatic in which at least one ring atom is a heteroatom. Heteroaryl encompasses 5- to 7-membered aromatic, monocyclic rings containing one or more, for example, from 1 to 4, or in certain embodiments, from 1 to 3, heteroatoms chosen from N, O, and S, with the remaining ring atoms being carbon; and bicyclic heterocycloalkyl rings containing one or more, for example, from 1 to 4, or in certain embodiments, from 1 to 3, heteroatoms chosen from N, O, and S, with the remaining ring atoms being carbon and wherein at least one heteroatom is present in an aromatic ring. For example, heteroaryl includes a 5- to 7-membered heterocycloalkyl, aromatic ring fused to a 5- to 7-membered cycloalkyl ring. For such fused, bicyclic heteroaryl ring systems wherein only one of the rings contains one or more heteroatoms, the point of attachment may be at the heteroaromatic ring or the cycloalkyl ring. In certain embodiments, when the total number of N, S, and O atoms in the heteroaryl group exceeds one, the heteroatoms are not adjacent to one another. In certain embodiments, the total number of N, S, and O atoms in the heteroaryl group is not more than two. In certain embodiments, the total number of N, S, and O atoms in the aromatic heterocycle is not more than one. Heteroaryl does not encompass or overlap with aryl as defined herein.

Examples of heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, and the like. In certain embodiments, a heteroaryl group is 5- to 20-membered heteroaryl, and in certain embodiments 5- to 10-membered heteroaryl. In certain embodiments, heteroaryl groups are derived from thiophene, pyrrole, benzothiophene, benzofuran, indole, pyridine, quinoline, imidazole, oxazole, and pyrazine.

“Heteroarylalkyl,” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature “heteroarylalkanyl,” “heteroarylalkenyl,” and “heterorylalkynyl” is used. In certain embodiments, a heteroarylalkyl group is a 6- to 30-membered heteroarylalkyl, e.g., the alkanyl, alkenyl, or alkynyl moiety of the heteroarylalkyl is 1- to 10-membered and the heteroaryl moiety is a 5- to 20-membered heteroaryl, and in certain embodiments, 6- to 20-membered heteroarylalkyl, e.g., the alkanyl, alkenyl, or alkynyl moiety of the heteroarylalkyl is 1- to 8-membered and the heteroaryl moiety is a 5- to 12-membered heteroaryl.

“N-oxide” refers to compounds in which a basic atom of either a heteroaromatic ring or tertiary amine is oxidized to give quaternary nitrogen bearing a positive formal charge and a bonded oxygen atom bearing a negative formal charge.

“Parent aromatic ring system” refers to an unsaturated cyclic or polycyclic ring system having a conjugated π (pi) electron system. Included within the definition of parent aromatic ring system are fused ring systems in which one or more of the rings are aromatic and one or more of the rings are saturated or unsaturated, such as, for example, fluorene, indane, indene, phenalene, etc. Examples of parent aromatic ring systems include, but are not limited to, aceanthrylene, acenaphthylene, acephenanthrylene, anthracene, azulene, benzene, chrysene, coronene, fluoranthene, fluorene, hexacene, hexaphene, hexylene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene, and the like.

“Parent heteroaromatic ring system” refers to a parent aromatic ring system in which one or more carbon atoms (and any associated hydrogen atoms) are independently replaced with the same or different heteroatom. Examples of heteroatoms to replace the carbon atoms include, but are not limited to, N, P, O, S, and Si, etc. Specifically included within the definition of parent heteroaromatic ring systems are fused ring systems in which one or more of the rings are aromatic and one or more of the rings are saturated or unsaturated, such as, for example, arsindole, benzodioxan, benzofuran, chromane, chromene, indole, indoline, xanthene, etc. Examples of parent heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, etc.

“Patient” refers to a mammal, for example, a human.

“Pharmaceutically acceptable” refers to approved or approvable by a regulatory agency of a federal or a state government, listed in the U.S. Pharmacopoeia, or listed in other generally recognized pharmacopoeia for use in mammals, including humans.

“Pharmaceutically acceptable salt” refers to a salt of a compound, which possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; and (2) salts formed when an acidic proton present in the parent compound is replaced by a metal ion, e.g., an alkali metal ion, an alkaline earth metal ion, or an aluminum ion; or coordinates with an organic base such as ethanolamine, diethanolamine, triethanolamine, N-methylglucamine, etc.

“Pharmaceutically acceptable solvate” refers to a molecular complex of a compound with one or more solvent molecules in a stoichiometric or non-stoichiometric amount. Such solvent molecules are those commonly used in the pharmaceutical arts, which are known to be innocuous to recipient, e.g., water, ethanol, and the like. A molecular complex of a compound or moiety of a compound and a solvent can be stabilized by non-covalent intra-molecular forces such as electrostatic forces, van der Waals forces, and hydrogen bonds. The term “hydrate” refers to a complex in which the one or more solvent molecules are water including monohydrates and hemi-hydrates.

“Pharmaceutically acceptable vehicle” refers to a pharmaceutically acceptable diluent, a pharmaceutically acceptable adjuvant, a pharmaceutically acceptable excipient, a pharmaceutically acceptable carrier, or a combination of any of the foregoing with which a compound provided by the present disclosure may be administered to a patient, which does not destroy the pharmacological activity thereof, and which is nontoxic when administered in doses sufficient to provide a therapeutically effective amount of the compound.

“Prodrug” refers to a derivative of an active compound (drug) that undergoes a transformation under the conditions of use, such as within the body, to release an active drug. For example, compounds of Formulae (I)-(VI) are levodopa prodrugs that can be metabolized within a patient's body to form the corresponding parent drug, levodopa. Prodrugs are frequently, but not necessarily, pharmacologically inactive until converted into the active drug. Prodrugs can be obtained by bonding a promoiety (defined herein), typically via a functional group, to a drug.

“Promoiety” refers to a group bonded to a drug, typically to a functional group of the drug, via bond(s) that are cleavable under specified conditions of use. The bond(s) between the drug and promoiety may be cleaved by enzymatic or non-enzymatic means. Under the conditions of use, for example following administration to a patient, the bond(s) between the drug and promoiety may be cleaved to release the parent drug. The cleavage of the promoiety may proceed spontaneously, such as via a hydrolysis reaction, or may be catalyzed or induced by another agent, such as by an enzyme, by light, by acid, or by a change of or exposure to a physical or environmental parameter, such as a change of temperature, pH, etc. The agent may be endogenous to the conditions of use, such as an enzyme present in the systemic circulation to which the prodrug is administered or the acidic conditions of the stomach, or the agent may be supplied exogenously. In certain embodiments, the drug is levodopa and the promoiety has the structure: 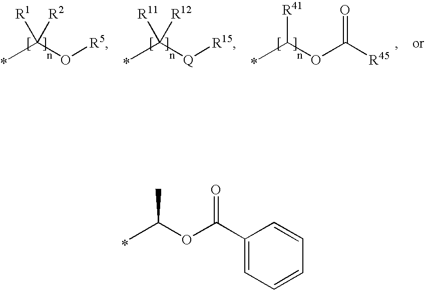
where n, R¹, R², R⁵, R¹¹, R₁₂, R¹⁵, R⁴¹, and R⁴⁵ are defined herein.

“Schizophrenia” means a group of neuropsychiatric disorders characterized by dysfunctions of the thinking process, such as delusions, hallucinations, and extensive withdrawal of the patient's interests form other people. Schizophrenia includes the subtypes of paranoid schizophrenia characterized by a preoccupation with delusions or auditory hallucinations, hebephrenic or disorganized schizophrenia characterized by disorganized speech, disorganized behavior, and flat or inappropriate emotions; catatonic schizophrenia dominated by physical symptoms such as immobility, excessive motor activity, or the assumption of bizarre postures; undifferentiated schizophrenia characterized by a combination of symptoms characteristic of the other subtypes; and residual schizophrenia in which a person is not currently suffering from positive symptoms but manifests negative and/or cognitive symptoms of schizophrenia (see DSM-IV-TR classifications 295.30 (Paranoid Type), 295.10 (Disorganized Type), 295.20 (Catatonic Type), 295.90 (Undifferentiated Type), and 295.60 (Residual Type) (Diagnostic and Statistical Manual of Mental Disorders, 4^{th} Edition, American Psychiatric Association, 297-319, 2005). Schizophrenia includes these and other closely associated psychotic disorders such as schizophreniform disorder, schizoaffective disorder, delusional disorder, brief psychotic disorder, shared psychotic disorder, psychotic disorder due to a general medical condition, substance-induced psychotic disorder, and unspecified psychotic disorders (DSM-IV-TR, 4^{th} Edition, pp. 297-344, American Psychiatric Association, 2005). Schizoaffective disorder characterized by symptoms of schizophrenia as well as mood disorder such as major depression, bipolar mania, or mixed mania, is included as a subtype of schizophrenia.

Schizophrenia symptoms can be classified as positive, negative, or cognitive. Positive symptoms of schizophrenia include delusion and hallucination, which can be measured using, for example, the Positive and Negative Syndrome Scale (PANSS) (see Kay et al., 1987, Schizophrenia Bulletin 13, 261-276). Negative symptoms of schizophrenia include affect blunting, anergia, alogia, and social withdrawal, which can be measured for example, using the Scales for the Assessment of Negative Symptoms (SANS) (see Andreasen, 1983, Scales for the Assessment of Negative Symptoms, Iowa City, Iowa). Cognitive symptoms of schizophrenia include impairment in obtaining, organizing, and using intellectual knowledge which can be measured using the Positive and Negative Syndrome Scale-cognitive subscale (PANSS-cognitive subscale) (Lindenmayer et al., J Nerv Ment Dis 1994, 182, 631-638) or by assessing the ability to perform cognitive tasks such as, for example, using the Wisconsin Card Sorting Test (see, e.g., Green et al., Am J Psychiatry 1992, 149, 162-67; and Koren et al., Schizophr Bull 2006, 32(2), 310-26).

“Substituted” refers to a group in which one or more hydrogen atoms are each independently replaced with the same or different substituent(s). Typical substituents include, but are not limited to, -M, —R³³, —O—, ═O, —OR³³, —SR³³, —S—, ═S, —NR³³R³⁴, ═NR³³, —CX₃, —CF₃, —CN, —OCN, —SCN, —NO, —NO₂, ═N₂, —N₃, —S(O)₂O⁻, —S(O)₂OH, —S(O)₂R³³, —OS(O₂)O⁻, —OS(O)₂R³³, —P(O)(O⁻)₂, —P(O)(OR³³)(O⁻), —OP(O)(OR³³)(OR³⁴), —C(O)R³³, —C(S)R³³, —C(O)OR³³, —C(O)NR³³R³⁴, —C(O)O⁻, —C(S)OR³³, —NR³⁵C(O)NR³³R³⁴, —NR³⁵C(S)NR³³R³⁴, —NR³⁵C(NR³³)NR³³R³⁴, and —C(NR³³)NR³³R³⁴, where each M is independently a halogen; each R³³ and R³⁴ are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, substituted heteroarylalkyl, —NR³⁵R³⁶, —C(O)R³⁵, and —S(O)₂R³⁵, or R³³ and R³⁴, together with the atom to which R³³ and R³⁴ are bonded form a cycloheteroalkyl or substituted cycloheteroalkyl ring; and R³⁵ and R³⁶ are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl, and substituted heteroarylalkyl.

In certain embodiments of the compounds of Formulae (I)-(VI), each of the one or more substituent groups is independently chosen from halo, —CN, —NO₂, —OH, C₁₋₆ alkyl, and C₁₋₆ alkoxy. In certain embodiments of compounds of Formulae (I)-(VI), each of the one or more substituent groups is independently chosen from halo, —OH, C₁₋₃ alkyl, and C₁₋₃ alkoxy.

“Sustained release” refers to release of a compound from a pharmaceutical composition dosage form at a rate effective to achieve a therapeutic or prophylactic concentration of the compound, or active metabolite thereof, in the systemic circulation of a patient over a prolonged period of time relative to that achieved by oral administration of an immediate release formulation of the same compound. In some embodiments, release of a compound occurs over a time period of at least about 4 hours, such as at least about 8 hours, at least about 12 hours, at least about 16 hours, at least about 20 hours, and in some embodiments, at least about 24 hours.

“Therapeutically effective amount” refers to the amount of a compound that, when administered to a subject for treating a disease or disorder, or at least one of the clinical symptoms of a disease or disorder, is sufficient to affect such treatment of the disease, disorder, or symptom. The therapeutically effective amount may vary depending, for example, on the compound, the disease, disorder, and/or symptoms of the disease, the severity of the disease, disorder, and/or symptoms thereof, the age, weight, and/or health of the patient to be treated, and the judgment of the prescribing physician. An appropriate therapeutically effective amount in any given instance may be readily ascertained by those skilled in the art or capable of determination by routine experimentation.

“Therapeutically effective dose” refers to a dose that provides effective treatment of a disease, disorder, or symptom in a patient. A therapeutically effective dose may vary from compound to compound, and from patient to patient, and can depend upon factors such as the condition of the patient and the route of delivery. A therapeutically effective dose may be determined in accordance with routine pharmacological procedures known to those skilled in the art.

“Treating” or “treatment” of any disease refers to arresting or ameliorating a disease or at least one of the clinical symptoms of a disease or disorder, reducing the risk of acquiring a disease or at least one of the clinical symptoms of a disease, reducing the development of a disease or at least one of the clinical symptoms of the disease or reducing the risk of developing a disease or at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to delaying the onset of the disease or at least one or more symptoms thereof in a patient which may be exposed to or predisposed to a disease or disorder even though that patient does not yet experience or display symptoms of the disease.

Treating schizophrenia encompasses treating one or more symptoms, positive, negative, cognitive, and other associated features, of schizophrenia. Examples of symptoms of schizophrenia include delusions, hallucinations, disorganized speech, affective flattening, alogia, anhedonia, inappropriate affect, dysphoric mood (in the form of, for example, depression, anxiety, and/or anger), and some indications of cognitive dysfunction.

Reference is now made in detail to certain embodiments of compounds, pharmaceutical compositions, dosage forms, and methods. The disclosed embodiments are not intended to be limiting of the claims. To the contrary, the claims are intended to cover all alternatives, modifications, and equivalents.

### Pharmaceutical Compositions

Pharmaceutical compositions provided by the present disclosure comprise at least one antipsychotic agent and at least one colonically absorbable form of levodopa. In certain embodiments, pharmaceutical compositions comprise at least one antipsychotic agent in an amount that is effective for treating a positive symptom of schizophrenia in a patient, and at least one colonically absorbable form of levodopa in an amount that is effective for treating a negative or cognitive symptom of schizophrenia in the patient, and that does not exacerbate or induce a positive symptom of schizophrenia in the patient. In certain embodiments, pharmaceutical compositions may include one or more antipsychotic agents and/or one or more colonically absorbable forms of levodopa. In certain embodiments, pharmaceutical compositions provided by the present disclosure may include a pharmaceutically acceptable vehicle. Pharmaceutically acceptable vehicles include diluents, adjuvants, excipients, and carriers.

In certain embodiments, antipsychotic agents may be any compound that has been shown to be useful or is believed to be useful in treating at least a positive symptom of schizophrenia. Antipsychotic agents useful in treating at least a positive symptom of schizophrenia include typical antipsychotic agents, atypical antipsychotic agents, and other antipsychotic agents that may or may not be classified as typical or atypical antipsychotic agents. In certain embodiments, an antipsychotic agent is a typical antipsychotic agent. In certain embodiments, an antipsychotic agent is a dopamine D2 receptor antagonist, which may be a selective dopamine D2 receptor antagonist or a partial dopamine D2 receptor antagonist. Typical antipsychotic agents are generally recognized as selective dopamine D2 receptor antagonists.

Antipsychotic agents useful in treating positive symptoms of schizophrenia include, but are not limited to, acetophenazine, alseroxylon, amitriptyline, aripiprazole, astemizole, benzquinamide, carphenazine, chlormezanone, chlorpromazine, chlorprothixene, clozapine, desipramine, droperidol, aloperidol, fluphenazine, flupenthixol, glycine, oxapine, mesoridazine, molindone, olanzapine, ondansetron, perphenazine, pimozide, prochlorperazine, procyclidine, promazine, propiomazine, quetiapine, remoxipride, reserpine, risperidone, sertindole, sulpiride, terfenadine, thiethylperzaine, thioridazine, thiothixene, trifluoperazine, triflupromazine, trimeprazine, and ziprasidone. Examples of typical antipsychotic agents useful for treating positive symptoms of schizophrenia include acetophenazine, chlorpromazine, chlorprothixene, droperidol, fluphenazine, haloperidol, loxapine, mesoridazine, methotrimeprazine, molindone, perphenazine, pimozide, raclopride, remoxipride, thioridazine, thiothixene, and trifluoperazine. Examples of atypical antipsychotic agents useful for treating positive symptoms of schizophrenia include aripiprazole, clozapine, olanzapine, quetiapine, risperidone, sertindole, and ziprasidone.

Other antipsychotic agents useful for treating positive symptoms of schizophrenia include amisulpride, balaperidone, blonanserin, butaperazine, carphenazine, eplavanserin, iloperidone, lamictal, onsanetant, paliperidone, perospirone, piperacetazine, raclopride, remoxipride, sarizotan, sonepiprazole, sulpiride, ziprasidone, and zotepine; serotonin and dopamine (5HT/D2) agonists such as asenapine and bifeprunox; neurokinin 3 antagonists such as talnetant and osanetant; AMPAkines such as CX-516, galantamine, memantine, modafinil, ocaperidone, and tolcapone; and α-amino acids such as D-serine, D-alanine, D-cycloserine, and N-methylglycine. Thus, antipsychotic agents include typical antipsychotic agents, atypical antipsychotic agents, and other compounds useful for treating schizophrenia in a patient, and particularly useful for treating the positive symptoms of schizophrenia.

Pharmaceutical compositions provided by the present disclosure comprise one or more colonically absorbable forms of levodopa, pharmaceutically acceptable salts thereof, or pharmaceutically acceptable solvates of any of the foregoing. In certain embodiments, pharmaceutical compositions comprise a colonically absorbable form of levodopa that exhibits an oral bioavailability that is at least 10 times greater than the oral bioavailability of levodopa when administered to a patient as a uniform liquid immediate release formulation. In certain embodiments, pharmaceutical compositions comprise a colonically absorbable form of levodopa that provides a levodopa plasma AUC in a patient following colonic administration that is at least two times greater than the levodopa plasma AUC in the patient following colonic administration of levodopa. Examples of colonically absorbable forms of levodopa include tight ion pairs of levodopa and prodrugs of levodopa. Colonically absorbable forms of levodopa include prodrugs, conjugates, and complexes. A promoiety covalently or non-covalently bonded to levodopa can enhance permeability through gastrointestinal epithelia via passive and/or active transport mechanisms, may control the release of levodopa in the gastrointestinal tract, and/or can inhibit enzymatic and chemical degradation of levodopa in the gastrointestinal tract. For colonically absorbable forms of levodopa in which the promoiety remains bonded to the levodopa molecule after absorption from the gastrointestinal tract, the promoiety may enhance permeability through other biological membranes, such as the blood-brain barrier, and/or may inhibit enzymatic and/or chemical degradation of levodopa in the systemic circulation.

Methods of determining the colonic absorbability of forms of levodopa are described in Xiang et al, U.S. Application Publication Nos. 2006/0020028 and 2005/0282891, and International Application Publication No. WO 2007/067495, each of which is incorporated by reference herein in its entirety.

In certain embodiments, a colonically absorbable form of levodopa is a tight-ion pair such as described by Wong et al., U.S. Application Publication No. 2005/0163850. Wong et al. disclose forming tight-ion pair complexes of generally hydrophobic compounds such as alkyl sulfates or fatty acids. The tight-ion pair complexes disclosed by Wong et al. are characterized by a generally hydrophobic exterior and are intended to be more stable than loose ion pairs in the presence of water rendering the complexes more likely to move through intestinal epithelial membranes by paricellular or active transport. Such tight-ion pair complexes may enhance absorption of drugs such as levodopa as well as prodrugs of levodopa in both the upper and lower gastrointestinal tract.

In certain embodiments, colonically absorbable forms of levodopa are levodopa prodrugs. Colonically absorbable prodrugs of levodopa can provide a greater oral bioavailability of levodopa relative to the oral bioavailability of levodopa when orally administered to a patient as a uniform liquid immediate release formulation. Examples of colonically absorbable levodopa prodrugs with enhanced oral bioavailability include, but are not limited to, bile acid prodrugs, peptide conjugates, and prodrugs in which levodopa is bonded to an amino acid or small peptide via a linkage.

Prodrugs are compounds in which a promoiety is typically covalently bonded to a drug. Following absorption from the gastrointestinal tract, the promoiety is cleaved to release the drug into the systemic circulation. While in the gastrointestinal tract, the promoiety can protect the drug from the harsh chemical environment, and can also facilitate absorption. Promoieties can be designed, for example, to enhance passive absorption, e.g., lipophilic promoieties, and/or enhance absorption via active transport mechanisms, e.g., substrate promoieties. In particular, active transporters differentially expressed in regions of the gastrointestinal tract can be preferentially targeted to enhance absorption in a particular region or regions of the gastrointestinal tract. For example, levodopa prodrugs may incorporate a promoiety that is a substrate of PEPT1 transporters expressed in the small intestine. Zerangue et al., U.S. Application Publication Nos. 2003/0017964 and 2005/0214853, each of which is incorporated by reference herein in its entirety, disclose methodologies for screening drugs, conjugates, or conjugate moieties, linked or linkable to drugs, for their capacity to be transported as substrates via the PEPT1 and PEPT2 transporters, which are known to be expressed in the human small intestine (see e.g., Fei et al., Nature 1964, 386, 563-566; and Miyamoto et al., Biochimica et Biophysica Acta 1996, 1305, 34-38). Zerangue et al, U.S. Application Publication No. 2003/0158254 also disclose several transporters expressed in the human colon including the sodium dependent multi-vitamin transporter (SMVT) and the monocarboxylate transporters MCT1 and MCT4, methods of identifying agents or conjugate moieties that are transporter substrates, and agents, conjugates, and conjugate moieties that can be screened for substrate activity. Zerangue et al further disclose compounds that can be screened that are variants of known transporter substrates such as bile salts or acids, steroids, ecosanoids, or natural toxins or analogs thereof, as described by Smith, Am. J Physiol 1987, 223, 974-978; Smith, Am J Physio. 1993, 252, G479-G484; Boyer, Proc Natl Acad Sci USA 1993, 90, 435-438; Fricker, Biochem J 1994, 299, 665-670; Ficker, Biochem J 1994, 299, 665-670; and Ballatori et al., Am J Physiol 2000, 278, G57-G63, and the linkage of drugs to conjugate moieties.

Conjugation to bile acids has been shown to enhance oral bioavailability of drugs. Bile acids are hydroxylated steroids that play a key role in digestion and absorption of fat and lipophilic vitamins. After synthesis in the liver, bile acids are secreted into bile and excreted by the gall bladder into the intestinal lumen where they emulsify and help solubilize lipophilic substances. Bile acids are conserved in the body by active uptake from the terminal ileum via the sodium-dependent transporter IBAT (or ASBT) and subsequent hepatic extraction by the transporter NTCP (or LBAT) located in the sinusoidal membrane of hepatocytes. Gallop et al. disclose prodrugs in which a drug is covalently bonded to a cleavable linker which in turn is covalently bonded to a promoiety, such as a bile acid or bile acid derivative that facilitates translocation of the conjugate across the intestinal epithelia via the bile acid transport system (see, Gallop et al., U.S. Pat. Nos. 6,984,634, 6,900,192, and 6,984,634; and U.S. Application Publication Nos. 2002/0099041, 2005/0272710, 2003/0130246, 2005/0148564, and 2005/0288228, each of which is incorporated by reference herein in its entirety). Following absorption via the bile acid transport system, the linker is cleaved to release the drug into the systemic circulation.

Another drug-modification method includes covalent bonding of drugs directly to an amino acid or polypeptide that stabilizes the active agent, primarily in the stomach, through conformational protection (see, e.g., Piccariello et al., U.S. Pat. No. 6,716,452, and U.S. Application Publication Nos. 2004/0127397 and 2004/0063628). Piccariello et al. disclose conjugates in which a drug, such as levodopa, can be covalently bonded directly to the N-terminus, the C-terminus, or an amino acid side chain of a carrier polypeptide. In certain applications, the polypeptide can stabilize the drug in the gastrointestinal tract through conformational protection and/or can act as a substrate for transporters such as PEPT transporters.

Certain active transporter proteins are expressed throughout the gastrointestinal tract. An active transporter refers to a membrane-bound protein that recognizes a substrate and affects the entry of the substrate into, or exit from a cell by carrier-mediated transport or receptor-mediated transport. Active transport includes movement of molecules across cellular membranes that is directly or indirectly dependent on an energy mediated process, such as for example, driven by ATP hydrolysis or an ion gradient, that occurs by facilitated diffusion mediated by interaction with specific transporter proteins, or that occurs through a modulated solute channel. For example, organic cation transporters such as OCTN1 and OCTN2 are expressed in the epithelial cells lining a human colon as well as in the small intestine.

Thus, levodopa prodrugs that act as substrates for one or more organic cation transporter(s) may exhibit increased active transporter-mediated absorption during the extended period of time that the compound passes through the gastrointestinal tract. Increased absorption and in particular colonic absorption of a levodopa prodrug may result in increased systemic bioavailability of the levodopa prodrug or levodopa metabolite over an extended period of time. Systemic bioavailability refers to the rate and extent of systemic exposure to a drug or an active metabolite thereof as reflected in the integrated systemic blood concentration over a period of time, also referred to as “area under the curve.”

In certain embodiments, colonically absorbable levodopa prodrugs are capable of absorption over a significant length of the gastrointestinal tract, including the large intestine, and in particular the colon. Such prodrugs may be incorporated into sustained release formulations including osmotic delivery devices to provide sustained systemic exposure to levodopa upon oral administration to a patient. Such prodrugs may be co-administered with a decarboxylase inhibitor such as carbidopa or benserazide, or a prodrug of thereof, and in some embodiments also formulated as sustained release compositions, with the carbidopa/levodopa prodrug compositions or benserazide/levodopa prodrug compositions together providing prolonged exposure to levodopa at levels necessary to effect sustained treatment of the negative and/or positive symptoms of schizophrenia. Certain embodiments include carbidopa prodrugs that block first-pass levodopa decarboxylation within the intestinal enterocytes either as the intact carbidopa prodrug or through generation of carbidopa from carbidopa prodrug cleavage within the enterocytes and which may be cleaved to provide carbidopa in the systemic circulation. Decarboxylase inhibitor/levodopa prodrug or decarboxylase inhibitor prodrug/levodopa prodrug sustained release compositions may also be administered together with inhibitors of catechol O-methyltransferase (COMT) such as entacapone or tolcapone, to further block peripheral clearance of levodopa.

In certain embodiments, colonically absorbable prodrugs of levodopa have the structure of Formula (I) as disclosed in Xiang et al., U.S. Application Publication No. U.S. Application Publication No. 2006/0020028, which is incorporated by reference herein in its entirety:

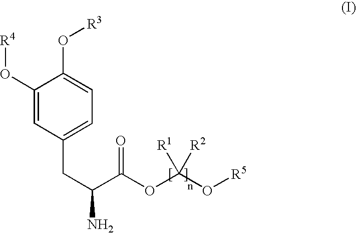

pharmaceutically acceptable salts of any of the foregoing, and pharmaceutically acceptable solvates of any of the foregoing, wherein

n is an integer chosen from 1 to 6;

each R¹ and R² is independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, halo, heteroalkyl, substituted heteroalkyl, heteroaryl, substituted heteroaryl, heteroarylalkyl, and substituted heteroarylalkyl, or when n is 1, then R¹ and R² together with the carbon atom to which R¹ and R² are bonded form a cycloalkyl, substituted cycloalkyl, cycloheteroalkyl, or substituted cycloheteroalkyl ring;

R³ and R⁴ are independently chosen from hydrogen, —C(O)OR⁷, —C(O)R⁷, and —(CR⁸R⁹)OC(O)R¹⁰;

R⁵ is chosen from alkyl, substituted alkyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl;

R⁷ is chosen from alkyl, substituted alkyl, cycloalkyl, substituted cycloalkyl, cycloheteroalkyl, substituted cycloheteroalkyl, aryl, substituted aryl, arylalkyl, substituted arylalkyl, heteroaryl, substituted heteroaryl, heteroarylalkyl, and substituted heteroarylalkyl;

R⁸ and R⁹ are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, and substituted heteroarylalkyl, or R⁸ and R⁹ together with the carbon atom to which R⁸ and R⁹ are bonded form a cycloalkyl, substituted cycloalkyl, cycloheteroalkyl, or substituted cycloheteroalkyl ring; and

R¹⁰ is chosen from hydrogen, alkyl, substituted alkyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl;

with the provisos that

when n is 2, and each of R¹, R², R³ and R⁴ is hydrogen, then R⁵ is not chosen from hydrogen, methyl, and phenyl;

when n is 3, and each of R¹, R², R³ and R⁴ is hydrogen, then R⁵ is not methyl; and

when n is an integer chosen from 1 to 6, and each of R¹, R³ and R⁴ is hydrogen, then R⁵ is not benzyl.

In certain embodiments of a colonically absorbable levodopa prodrug of Formula (I):

n is an integer chosen from 1 to 6;

each R¹ and R² is independently chosen from hydrogen and phenyl, which is optionally substituted with one or more substituents independently chosen from halo, —CN, —NO₂, —OH, C₁₋₆ alkyl, and C₁₋₆ alkoxy, or when n is 1, then R¹ and R² together with the carbon atom to which R¹ and R² are bonded form a cycloalkyl, substituted cycloalkyl, cycloheteroalkyl, or substituted cycloheteroalkyl ring;

R³ and R⁴ are independently chosen from hydrogen, —C(O)OR⁷, —C(O)R⁷, and —(CR⁸R⁹)OC(O)R¹⁰;

R⁵ is chosen from alkyl, substituted alkyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl;

R⁷ is chosen from alkyl, substituted alkyl, cycloalkyl, substituted cycloalkyl, cycloheteroalkyl, substituted cycloheteroalkyl, aryl, substituted aryl, arylalkyl, substituted arylalkyl, heteroaryl, substituted heteroaryl, heteroarylalkyl, and substituted heteroarylalkyl;

R⁸ and R⁹ are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, and substituted heteroarylalkyl, or R⁸ and R⁹ together with the carbon atom to which R⁸ and R⁹ are bonded form a cycloalkyl, substituted cycloalkyl, cycloheteroalkyl, or substituted cycloheteroalkyl ring;

R¹⁰ is chosen from hydrogen, alkyl, substituted alkyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl; and

wherein each substituent group is independently chosen from halo, —CN, —NO₂, —OH, C₁₋₆ alkyl, and C₁₋₆ alkoxy,

with the provisos that

when n is 2, and each of R¹, R², R³ and R⁴ is hydrogen, then R⁵ is not chosen from methyl and phenyl;

when n is 3, and each of R¹, R², R³ and R⁴ is hydrogen, then R⁵ is not methyl; and

when n is an integer chosen from 1 to 6, and each of R¹, R², R³ and R⁴ is hydrogen, then R⁵ is not benzyl.

In certain embodiments, colonically absorbable prodrugs of levodopa may be chosen from any of the genera or species of compounds of Formula (I) disclosed in Xiang et al., U.S. Application Publication No. 2006/0020028, which is incorporated by reference herein in its entirety. For example, in certain embodiments, colonically absorbable prodrugs of levodopa of Formula (I) are chosen from:
2-(4-fluorophenoxy)ethyl (2S)-2-amino-3-(3,4-dihydroxyphenyl)propanoate;
2-(4-chlorophenoxy)ethyl (2S)-2-amino-3-(3,4-dihydroxyphenyl)propanoate;
2-(4-methylphenoxy)ethyl (2S)-2-amino-3-(3,4-dihydroxyphenyl)propanoate;
2-(4-methoxyphenoxy)ethyl (2S)-2-amino-3-(3,4-dihydroxyphenyl)propanoate;
2-(2-fluorophenoxy)ethyl (2S)-2-amino-3-(3,4-dihydroxyphenyl)propanoate;
2-(4-butylphenoxy)ethyl (2S)-2-amino-3-(3,4-dihydroxyphenyl)propanoate;
2-(3-fluorophenoxy)ethyl (2S)-2-amino-3-(3,4-dihydroxyphenyl)propanoate;
2-(4-tert-butylphenoxy)ethyl (2S)-2-amino-3-(3,4-dihydroxyphenyl)propanoate;
2-(4-isopropylphenoxy)ethyl (2S)-2-amino-3-(3,4-dihydroxyphenyl)propanoate;
2-(4-ethylphenoxy)ethyl (2S)-2-amino-3-(3,4-dihydroxyphenyl)propanoate;
2-(2,4-dimethylphenoxy)ethyl (2S)-2-amino-3-(3,4-dihydroxyphenyl)propanoate;
2-(3,4-dimethylphenoxy)ethyl (2S)-2-amino-3-(3,4-dihydroxyphenyl)propanoate;
2-(4-fluorophenoxy)ethyl (2S)-2-amino-3-(3,4-diethoxycarbonyloxyphenyl)propanoate;
3-phenoxypropyl (2S)-2-amino-3-(3,4-dihydroxyphenyl)propanoate;
3-(4-fluorophenoxy)propyl (2S)-2-amino-3-(3,4-dihydroxyphenyl)propanoate;
(2R)-2-(4-fluorophenoxy)isopropyl (2S)-2-amino-3-(3,4-dihydroxyphenyl)propanoate;
(2S)-2-(4-fluorophenoxy)isopropyl (2S)-2-amino-3-(3,4-dihydroxyphenyl)propanoate;

pharmaceutically acceptable salts of any of the foregoing, and pharmaceutically acceptable solvates of any of the foregoing.

In certain embodiments, colonically absorbable prodrugs of levodopa have the structure of Formula (II) as disclosed in Xiang et al., U.S. Application Publication No. 2005/0282891, which is incorporated by reference herein in its entirety:

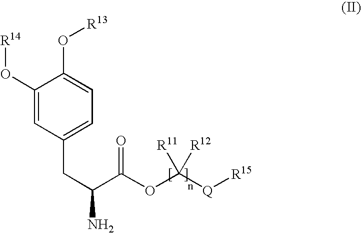

pharmaceutically acceptable salts of any of the foregoing, and pharmaceutically acceptable solvates of any of the foregoing, wherein

Q is chosen from —X—CO— and —CO—X—;

X is chosen from —O— and —NR¹⁶—;

n is an integer chosen from 2 to 4;

each R¹¹ and R¹² is independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, halo, heteroalkyl, substituted heteroalkyl, heteroaryl, substituted heteroaryl, heteroarylalkyl, and substituted heteroarylalkyl;

R¹³ and R¹⁴ are independently chosen from hydrogen, —C(O)OR¹⁷, —C(O)R¹⁷, and —(CR¹⁸R¹⁹)OC(O)R²⁰;

R¹⁵ is chosen from hydrogen, alkyl, substituted alkyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl; and when Q is —X—CO—, R¹⁵ is further chosen from alkoxy, substituted alkoxy, cycloalkoxy, and substituted cycloalkoxy;

R¹⁶ is chosen from hydrogen, alkyl, substituted alkyl, aryl, substituted aryl, arylalkyl, and substituted arylalkyl;

R¹⁷ is chosen from alkyl, substituted alkyl, cycloalkyl, substituted cycloalkyl, cycloheteroalkyl, substituted cycloheteroalkyl, aryl, substituted aryl, arylalkyl, substituted arylalkyl, heteroaryl, substituted heteroaryl, heteroarylalkyl, and substituted heteroarylalkyl;

R¹⁸ and R¹⁹ are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, and substituted heteroarylalkyl, or R¹⁸ and R¹⁹ together with the carbon atom to which R¹⁸ and R¹⁹ are bonded form a cycloalkyl, substituted cycloalkyl, cycloheteroalkyl, or substituted cycloheteroalkyl ring; and

R²⁰ is chosen from hydrogen, alkyl, substituted alkyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl;

with the proviso that the compound of Formula (II) is not derived from 1,3-dihexadecanoylpropane-1,2,3-triol.

In certain embodiments of a colonically absorbable levodopa prodrug of Formula (II):

Q is chosen from —X—CO— and —CO—X—;

X is chosen from —O— and —NH—;

n is an integer chosen from 2 to 4;

each R¹¹ and R¹² is independently chosen from hydrogen, methyl, ethyl, propyl, isopropyl, butyl, isobutyl, sec-butyl, tert-butyl, cyclopropyl, cyclobutyl, cyclopentyl, cyclohexyl, benzyl, alkanyl, substituted alkanyl, arylalkanyl, aryl, substituted aryl, heteroaryl, substituted heteroaryl, —OH, C₁₋₆ alkyl, and substituted C₁₋₆ alkyl;

R¹³ and R¹⁴ are independently chosen from hydrogen, —C(O)OR¹⁷, —C(O)R¹⁷, and —(CR¹⁸R¹⁹)OC(O)R²⁰;

R¹⁵ is chosen from hydrogen, alkyl, substituted alkyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl; and when Q is —X—CO—, R¹⁵ is further chosen from alkoxy, substituted alkoxy, cycloalkoxy, and substituted cycloalkoxy;

R¹⁷ is chosen from alkyl, substituted alkyl, cycloalkyl, substituted cycloalkyl, cycloheteroalkyl, substituted cycloheteroalkyl, aryl, substituted aryl, arylalkyl, substituted arylalkyl, heteroaryl, substituted heteroaryl, heteroarylalkyl, and substituted heteroarylalkyl;

R¹⁸ and R¹⁹ are independently chosen from hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroaryl, substituted heteroaryl, heteroarylalkyl, and substituted heteroarylalkyl, or R¹⁸ and R¹⁹ together with the carbon atom to which R¹⁸ and R¹⁹ are bonded form a cycloalkyl, substituted cycloalkyl, cycloheteroalkyl, or substituted cycloheteroalkyl ring;

R²⁰ is chosen from hydrogen, alkyl, substituted alkyl, aryl, substituted aryl, arylalkyl, substituted arylalkyl, cycloalkyl, substituted cycloalkyl, heteroalkyl, substituted heteroalkyl, cycloheteroalkyl, substituted cycloheteroalkyl, heteroaryl, substituted heteroaryl, heteroarylalkyl, and substituted heteroarylalkyl; and

wherein each substituent group is independently chosen from halo, —CN, —NO₂, —OH, C₁₋₆ alkyl, and C₁₋₆ alkoxy;

with the proviso that the compound of Formula (II) is not derived from 1,3-dihexadecanoylpropane-1,2,3-triol.

In certain embodiments, colonically absorbable prodrugs of levodopa may be chosen from any of the genera or species of compounds of Formula (II) disclosed in Xiang et al., U.S. Application Publication No. 2005/0282891, which is incorporated by reference herein in its entirety. For example, in certain embodiments, colonically absorbable prodrugs of levodopa is a compound of Formula (III):

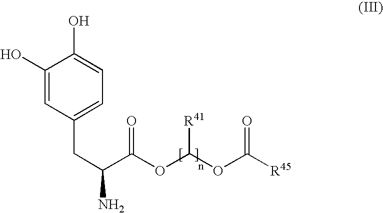

pharmaceutically acceptable salts of any of the foregoing, and pharmaceutically acceptable solvates of any of the foregoing, wherein

n is an integer chosen from 2 to 4;

each R⁴¹ is independently chosen from hydrogen, a straight chain C₁₋₃ alkyl, and a branched C₁₋₃ alkyl;

R⁴⁵ is chosen from phenyl and substituted phenyl wherein each of the one or more of the substituents is independently chosen from halo, —CN, —NO₂, —OH, C₁₋₆ alkyl, and C₁₋₆ alkoxy.

In certain embodiments, colonically absorbable prodrugs of levodopa of Formula (II) are chosen from:
2-phenylcarbonyloxyethyl (2S)-2-amino-3-(3,4-dihydroxyphenyl)propanoate;
2-(4-fluorophenylcarbonyloxy)ethyl (2S)-2-amino-3-(3,4-dihydroxyphenyl)propanoate;
3-phenylcarbonyloxypropyl (2S)-2-amino-3-(3,4-dihydroxyphenyl)propanoate;
3-(4-fluorophenylcarbonyloxy)propyl (2S)-2-amino-3-(3,4-dihydroxyphenyl)propanoate;
2-acetyloxyethyl (2S)-2-amino-3-(3,4-dihydroxyphenyl)propanoate;
(2R)-2-phenylcarbonyloxypropyl (2S)-2-amino-3-(3,4-dihydroxyphenyl)propanoate;
(2S)-2-phenylcarbonyloxypropyl (2S)-2-amino-3-(3,4-dihydroxyphenyl)propanoate;
(2R)-2-(4-fluorophenylcarbonyloxy)propyl (2S)-2-amino-3-(3,4-dihydroxyphenyl)propanoate;
(2S)-2-(4-fluorophenylcarbonyloxy)propyl (2S)-2-amino-3-(3,4-dihydroxyphenyl)propanoate;
(1R)-1-methyl-2-phenylcarbonyloxyethyl (2S)-2-amino-3-(3,4-dihydroxyphenyl)propanoate;
(1S)-1-methyl-2-phenylcarbonyloxyethyl (2S)-2-amino-3-(3,4-dihydroxyphenyl)propanoate;
(1R)-1-methyl-2-(4-fluorophenylcarbonyloxy)ethyl (2S)-2-amino-3-(3,4-dihydroxyphenyl)propanoate;
(1S)-1-methyl-2-(4-fluorophenylcarbonyloxy)ethyl (2S)-2-amino-3-(3,4-dihydroxyphenyl)propanoate;
(1R,2R)-1-methyl-2-phenylcarbonyloxypropyl (2S)-2-amino-3-(3,4-dihydroxyphenyl)propanoate;
(1S,2S)-1-methyl-2-phenylcarbonyloxypropyl (2S)-2-amino-3-(3,4-dihydroxyphenyl)propanoate;
(1R,2R)-1-methyl-2-(4-fluorophenylcarbonyloxy)propyl (2S)-2-amino-3-(3,4-dihydroxyphenyl)propanoate;
(1S,2S)-1-methyl-2-(4-fluorophenylcarbonyloxy)propyl (2S)-2-amino-3-(3,4-dihydroxyphenyl)propanoate;
3-(4-methoxyphenylcarbonyloxy)propyl (2S)-2-amino-3-(3,4-dihydroxyphenyl)propanoate;
3-(2-hydroxyphenylcarbonyloxy)propyl (2S)-2-amino-3-(3,4-dihydroxyphenyl)propanoate;
3-(4-hydroxyphenylcarbonyloxy)propyl (2S)-2-amino-3-(3,4-dihydroxyphenyl)propanoate;
2-hydroxy-3-phenylcarbonyloxypropyl (2S)-2-amino-3-(3,4-dihydroxyphenyl)propanoate;
(2R)-2-(2-hydroxyphenylcarbonyloxy)propyl (2S)-2-amino-3-(3,4-dihydroxyphenyl)propanoate;
(2R)-2-(4-hydroxyphenylcarbonyloxy)propyl (2S)-2-amino-3-(3,4-dihydroxyphenyl)propanoate;
(2R)-2-(4-methoxyphenylcarbonyloxy)propyl (2S)-2-amino-3-(3,4-dihydroxyphenyl)propanoate;
2-[(2-hydroxyphenyl)carbonylamino]ethyl (2S)-2-amino-3-(3,4-dihydroxyphenyl)propanoate;
2(R)-(3-pyridylcarbonyloxy)propyl (2S)-2-amino-3-(3,4-dihydroxyphenyl)propanoate;
2(S)-(3-pyridylcarbonyloxy)propyl (2S)-2-amino-3-(3,4-dihydroxyphenyl)propanoate;
2(R)-(4-pyridylcarbonyloxy)propyl (2S)-2-amino-3-(3,4-dihydroxyphenyl)propanoate;
2(S)-(4-pyridylcarbonyloxy)propyl (2S)-2-amino-3-(3,4-dihydroxyphenyl)propanoate;
2(R)-(2-ethoxy-3-pyridylcarbonyloxy)propyl (2S)-2-amino-3-(3,4-dihydroxyphenyl)propanoate;
2(S)-(2-ethoxy-3-pyridylcarbonyloxy)propyl (2S)-2-amino-3-(3,4-dihydroxyphenyl)propanoate;
2(R)-(2-methyl-5-pyridylcarbonyloxy)propyl (2S)-2-amino-3-(3,4-dihydroxyphenyl)propanoate;
2(S)-(2-methyl-5-pyridylcarbonyloxy)propyl (2S)-2-amino-3-(3,4-dihydroxyphenyl)propanoate;

pharmaceutically acceptable salts of any of the foregoing, and pharmaceutically acceptable solvates of any of the foregoing.

In certain embodiments, a colonically absorbable prodrug of levodopa is (2R)-2-phenylcarbonyloxypropyl (2S)-2-amino-3-(3,4-dihydroxyphenyl)propanoate mesylate, Formula (IV): 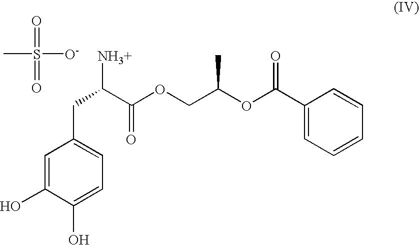
or crystalline form thereof as disclosed in Xiang et al., U.S. application Ser. No. 11/634,354, which is incorporated by reference herein in its entirety.

In certain embodiments, colonically absorbable prodrugs of levodopa may be chosen from any of the genera or species of compounds of Formula (IV) disclosed in Xiang et al., U.S. application Ser. No. 11/634,354.

In certain embodiments, colonically absorbable prodrugs of levodopa may be chosen from any of the genera or species of compounds of Formula (V) disclosed in Xiang et al., U.S. Provisional Application No. 60/876,148, filed Dec. 21, 2006, and the related provisional application filed Sep. 7, 2007, each of which is incorporated by reference herein in its entirety. For example, in certain embodiments, colonically absorbable prodrugs of levodopa is a compound of Formula (V):

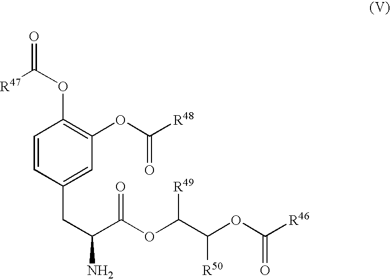

stereoisomers thereof, pharmaceutically acceptable salts of any of the foregoing, or pharmaceutically acceptable solvates of any of the foregoing, wherein:

R⁴⁶ is chosen from C₁₋₈ alkyl, substituted C₁₋₈ alkyl, C₁₋₈ alkoxy, substituted C₁₋₈ alkoxy, C₃₋₇ cycloalkyl, substituted C₃₋₇ cycloalkyl, C₃₋₇ cycloalkoxy, substituted C₃₋₇ cycloalkoxy, phenyl, substituted phenyl, phenyloxy, and substituted phenoxy;

R⁴⁷ and R⁴⁸ are independently chosen from C₁₋₈ alkyl, substituted C₁₋₈ alkyl, C₁₋₈ alkoxy, substituted C₁₋₈ alkoxy, C₃₋₇ cycloalkyl, and substituted C₃₋₇ cycloalkyl;

R⁴⁹ and R⁵⁰ are independently chosen from hydrogen, C₁₋₈ alkyl, and substituted C₁₋₈ alkyl;

wherein each substituent group is independently chosen from halogen, —OH, —COOH, —CN, —CF₃, ═O, —NO₂, C₁₋₃ alkoxy, C₁₋₃ alkyl, and —NR⁶⁰₂ wherein each R⁶⁰ is independently chosen from hydrogen and C₁₋₃ alkyl.

In certain embodiments, colonically absorbable prodrugs of levodopa of Formula (V) are chosen from:
(1R)-1-methyl-2-phenylcarbonyloxyethyl (2S)-3-(3,4-dihydroxyphenyl)-2-[(tert-butoxy)carbonylamino]propanoate;
(1R)-1-methyl-2-phenylcarbonyloxyethyl (2S)-2-amino-3-[3,4-bis(ethoxycarbonyloxy)phenyl]propanoate hydrochloride;
(1R)-1-methyl-2-phenylcarbonyloxyethyl (2S)-2-amino-3-[3,4-bis(isopropoxycarbonyloxy)phenyl]propanoate hydrochloride;
(1R)-1-methyl-2-phenylcarbonyloxyethyl (2S)-2-amino-3-[3,4-bis(2-methylpropanoyloxy)phenyl]propanoate hydrochloride;
(1R)-1-methyl-2-phenylcarbonyloxyethyl (2S)-2-[(tert-butoxy)carbonylamino]-3-[3,4-bis(2,2-dimethylpropanoyloxy)phenyl]propanoate;
(1R)-2-acetyloxy-1-methylethyl (2S)-2-amino-3-[3,4-bis(ethoxycarbonyloxy)phenyl]propanoate hydrochloride;
(1R)-2-acetyloxy-1-methylethyl (2S)-2-amino-3-[3,4-bis(ethoxycarbonyloxy)phenyl]propanoate hydrogen fumarate;
(1R)-1-methyl-2-(2-methylpropanoyloxy)ethyl (2S)-2-amino-3-[3,4-bis(ethoxycarbonyloxy)phenyl]propanoate hydrochloride;
(1R)-1-methyl-2-phenylcarbonyloxyethyl (2S)-2-amino-3-[3,4-bis(ethoxycarbonyloxy)phenyl]propanoate hydrochloride;
(1R)-2-ethoxycarbonyloxy-1-methylethyl (2S)-2-amino-3-[3,4-bis(ethoxycarbonyloxy)phenyl]propanoate hydrochloride;
(1R)-2-acetyloxy-1-methylethyl (2S)-2-amino-3-[3,4-bis(methylethoxycarbonyloxy)phenyl]propanoate hydrochloride;
(1R)-1-methyl-2-phenylcarbonyloxyethyl (2S)-2-amino-3-[3,4-bis(isopropoxycarbonyloxy)phenyl]propanoate hydrochloride;
(1R)-2-ethoxycarbonyloxy-1-methylethyl (2S)-2-amino-3-[3,4-bis(isopropoxycarbonyloxy)phenyl]propanoate hydrochloride;
(1R)-2-isopropoxycarbonyloxy-1-methylethyl (2S)-2-amino-3-(3,4-bis(isopropoxycarbonyloxy)phenyl)propanoate hydrochloride;
(1R)-1-methyl-2-phenylcarbonyloxyethyl (2S)-2-amino-3-[3,4-bis(isobutanoyloxy)phenyl]propanoate hydrochloride;
(1R)-1-methyl-2-phenylcarbonyloxyethyl (2S)-2-amino-3-[3,4-bis(2,2-dimethylpropanoyloxy)phenyl]propanoate hydrochloride;
(1R,2R)-2-acetoxy-1-methylpropyl (2S)-2-amino-3-[3,4-bis(ethoxycarbonyloxy)phenyl]propanoate hydrochloride;
(1R,2R)-1-methyl-2-(2-methylpropanoyloxy)propyl (2S)-2-amino-3-[3,4-bis(ethoxycarbonyloxy)phenyl]propanoate hydrochloride;
(1R,2R)-2-isobutoxycarbonyloxy-1-methylpropyl (2S)-2-amino-3-[3,4-bis(ethoxycarbonyloxy)phenyl]propanoate hydrochloride;
(1R,2R)-2-isopropoxycarbonyloxy-1-methylpropyl (2S)-2-amino-3-[3,4-bis(ethoxycarbonyloxy)phenyl]propanoate hydrochloride;
(1R,2R)-1-methyl-2-pentyloxycarbonyloxypropyl (2S)-2-amino-3-[3,4-bis(ethoxycarbonyloxy)phenyl]propanoate hydrochloride;
(1R,2R)-2-hexyloxycarbonyloxy-1-methylpropyl (2S)-2-amino-3-[3,4-bis(ethoxycarbonyloxy)phenyl]propanoate hydrochloride;
(1R,2R)-2-acetoxy-1-methylpropyl (2S)-2-amino-3-[3,4-bis(isopropoxycarbonyloxy)phenyl]propanoate hydrochloride;
(1R,2R)-1-methyl-2-(2-methylproparoyloxy)propyl (2S)-2-amino-3-[3,4-bis(isopropoxycarbonyloxy)phenyl]propanoate hydrochloride;
(2R)-2-acetyloxypropyl (2S)-2-amino-3-[3,4-bis(ethoxycarbonyloxy)phenyl]propanoate hydrochloride;
(2R)-2-acetyloxypropyl (2S)-2-amino-3-[3,4-bis(methylethoxycarbonyloxy)phenyl]propanoate hydrochloride;
(2R)-2-phenylcarbonyloxypropyl (2S)-2-amino-3-[3,4-bis(ethoxycarbonyloxy)phenyl]propanoate hydrochloride;
(2R)-2-phenylcarbonyloxypropyl (2S)-2-amino-3-(3,4-bis(methylethoxycarbonyloxy)phenyl]propanoate hydrochloride;

pharmaceutically acceptable salts of any of the foregoing, and pharmaceutically acceptable solvates of any of the foregoing.

In certain embodiments of a compound of Formula (V), the compound is a salt chosen from the hydrochloride salt and the fumarate salt.

In certain embodiments, colonically absorbable prodrugs of levodopa may be chosen from any of the genera or species of compounds of Formula (VI) disclosed in Xiang et al., U.S. Provisional Application No. 60/876,144, filed Dec. 21, 2006 and the related provisional application filed Sep. 7, 2007, each of which is incorporated by reference herein in its entirety. For example, in certain embodiments, colonically absorbable prodrugs of levodopa is a compound of Formula (VI):

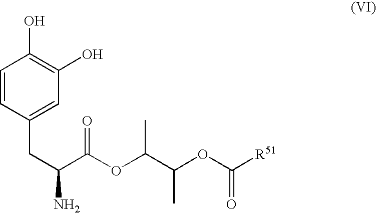

pharmaceutically acceptable salts of any of the foregoing, or pharmaceutically acceptable solvates of any of the foregoing, wherein:

R⁵¹ is chosen from C₁₋₈ alkyl, substituted C₁₋₈ alkyl, C₁₋₈ alkoxy, substituted C₁₋₈ alkoxy, C₃₋₇ cycloalkyl, substituted C₃₋₇ cycloalkyl, C₃₋₇ cycloalkoxy, substituted C₃₋₇ cycloalkoxy, C₆₋₁₂ aryl, and C₆₋₁₂ aryloxy;

wherein each substituent group is independently chosen from halogen, —OH, —COOH, —CN, —CF₃, ═O, —NO₂, C₁₋₃ alkoxy, C₁₋₃ alkyl, and —NR⁶¹₂ wherein each R⁶¹ is independently chosen from hydrogen and C₁₋₃ alkyl.

In certain embodiments, colonically absorbable prodrugs of levodopa of Formula (VI) are chosen from:
(1R,2R)-1-methyl-2-phenylcarbonyloxypropyl (2S)-2-amino-3-(3,4-dihydroxyphenyl)propanoate hydrochloride;
(1S,2S)-1-methyl-2-phenylcarbonyloxypropyl(2S)-2-amino-3-(3,4-dihydroxyphenyl)propanoate hydrochloride;
(1R,2R)-1-methyl-2-(4-fluorophenylcarbonyloxy)propyl (2S)-2-amino-3-(3,4-dihydroxyphenyl)propanoate hydrochloride;
(1S,2S)-1-methyl-2-(4-fluorophenylcarbonyloxy)propyl (2S)-2-amino-3-(3,4-dihydroxyphenyl)propanoate hydrochloride;
(1R,2R)-2-acetyloxy-1-methylpropyl (2S)-2-amino-3-(3,4-dihydroxyphenyl)propanoate hydrochloride;
(1R,2R)-1-methyl-2-(2-methylpropanoyloxy)propyl (2S)-2-amino-3-(3,4-dihydroxyphenyl)propanoate hydrochloride;
(1R,2R)-1-methyl-2-(2-methylpropanoyloxy)propyl (2S)-2-amino-3-(3,4-dihydroxyphenyl)propanoate mesylate;
(1R,2R)-1-methyl-2-phenylcarbonyloxypropyl (2S)-2-amino-3-(3,4-dihydroxyphenyl)propanoate hydrochloride;
(1R,2R)-1-methyl-2-phenylcarbonyloxypropyl (2S)-2-amino-3-(3,4-dihydroxyphenyl)propanoate mesylate;
(1R,2R)-2-ethoxycarbonyloxy-1-methylpropyl (2S)-2-amino-3-(3,4-dihydroxyphenyl)propanoate hydrochloride;
(1R,2R)-2-isopropoxycarbonyloxy-1-methylpropyl (2S)-2-amino-3-(3,4-dihydroxyphenyl)propanoate hydrochloride;
(1R,2R)-1-methyl-2-(2-methylpropoxycarbonyloxy)propyl (2S)-2-amino-3-(3,4-dihydroxyphenyl)propanoate, hydrochloride;
(1R,2R)-1-methyl-2-pentyloxycarbonyloxypropyl (2S)-2-amino-3-(3,4-dihydroxyphenyl)propanoate hydrochloride;

pharmaceutically acceptable salts of any of the foregoing, and pharmaceutically acceptable solvates of any of the foregoing.

In certain embodiments of compounds of Formula (VI), the compound is a salt chosen from the hydrochloride salt and the mesylate salt.

In certain embodiments, colonically absorbable levodopa prodrugs are chosen from compounds of Formula (I), compounds of Formula (II), compounds of Formula (III), compounds of Formula (IV), compounds of Formula (V), compounds of Formula (VI), pharmaceutically acceptable salts of any of the foregoing, pharmaceutically acceptable solvates of any of the foregoing, and combinations of any of the foregoing. In certain embodiments, colonically absorbable levodopa prodrugs provide a levodopa plasma AUC in a patient following colonic administration that is at least two times greater than the levodopa plasma AUC in the patient following colonic administration of an equivalent amount of levodopa in an equivalent dosage form.

Once absorbed, levodopa is rapidly converted to dopamine by L-aromatic amino acid decarboxylase (AADC) in the peripheral tissues (e.g., intestines and liver) and intestinal metabolism of levodopa is the major source of first pass loss of the drug. In human patients, only about 1% of an orally administered dose of levodopa reaches the central nervous system intact, following transport across the blood-brain barrier by the neutral amino acid transporter. For this reason, levodopa is normally co-administered with a drug designed to inhibit its peripheral decarboxylation such as carbidopa or benserazide. When administered with carbidopa or benserazide, the plasma intact levodopa amount increases and thus more levodopa becomes available to be transported into the central nervous system where it is converted to dopamine. Carbidopa and benserazide themselves do not cross the blood-brain barrier to a significant extent, and therefore do not inhibit the required conversion of levodopa to dopamine in the brain.

Thus, the half-life of levodopa is prolonged and its bioavailability increased by co-administration with a decarboxylase inhibitor such as carbidopa or benserazide. Both drugs have relatively short half-lives of less than about 2 hours. Any method of sustained delivery of levodopa to the systemic circulation therefore requires a sufficient level of a decarboxylase inhibitor such as carbidopa to continuously inhibit peripheral decarboxylation of levodopa. In order to avoid the need for frequent (more than twice per day) dosing of levodopa and carbidopa, it is desirable to deliver both levodopa and carbidopa (or prodrug thereof) in a sustained manner. Thus, in certain embodiments, pharmaceutical compositions provided by the present disclosure comprise a decarboxylase inhibitor such as carbidopa or benserazide.

In certain embodiments, pharmaceutical compositions provided by the present disclosure comprise, or a form of levodopa may be administered together with, a catechol-O-methyl transferase (COMT) inhibitor to further block peripheral clearance of levodopa. Examples of COMT inhibitors include entacapone, tolcapone, prodrugs of entacapone, prodrugs of tolcapone, pharmaceutically acceptable salts of any of the foregoing, pharmaceutically acceptable solvates of any of the foregoing, and combinations of any of the foregoing.

Pharmaceutical compositions provided by the present disclosure may be produced using standard procedures (see, e.g., Remington's The Science and Practice of Pharmacy, 21st Edition, Lippincott, Williams & Wilcox, 2005). The pharmaceutical compositions may be manufactured by means of conventional mixing, dissolving, granulating, dragee-making, levigating, emulsifying, encapsulating, entrapping, and lyophilizing processes. Pharmaceutical compositions may be formulated in a conventional manner using one or more physiologically acceptable carriers, diluents, excipients, and auxiliaries, which facilitate processing of compounds disclosed herein into preparations, which may be used pharmaceutically. Proper formulation may depend, in part, on the route of administration.

Pharmaceutical compositions provided by the present disclosure may provide therapeutic levels of an antipsychotic agent upon administration to a patient. In certain embodiments, antipsychotic agents may be any of the forms disclosed herein for levodopa, including a tight-ion pair or prodrug of the antipsychotic agent. In certain embodiments, antipsychotic agents may be in a form that exhibits enhanced colonic absorption relative to the antipsychotic agent itself.

Pharmaceutical compositions provided by the present disclosure may provide therapeutic levels of levodopa upon administration to a patient. The promoiety of a levodopa prodrug may be cleaved in vivo either chemically and/or enzymatically to release levodopa. One or more enzymes present in the stomach, intestinal lumen, intestinal tissue, blood, liver, brain, or any other suitable tissue of a mammal may enzymatically cleave the promoiety of the administered prodrugs. For example, the promoiety may be cleaved prior to absorption by the gastrointestinal tract (e.g., within the stomach or intestinal lumen) and/or after absorption by the gastrointestinal tract (e.g., in intestinal tissue, blood, liver, or other suitable tissue of a mammal). In certain embodiments, levodopa remains conjugated to the promoiety during transit across the intestinal mucosal barrier to provide protection from presystemic metabolism. In certain embodiments, a levodopa prodrug is essentially not metabolized to release levodopa within enterocytes, but is metabolized to levodopa within the systemic circulation. Cleavage of the promoiety of a levodopa prodrug after absorption by the gastrointestinal tract may allow the prodrugs to be absorbed into the systemic circulation by active transport, passive diffusion, or by a combination of both active and passive processes.

In certain embodiments, pharmaceutical compositions include an adjuvant that facilitates absorption of the at least one antipsychotic agent and/or at least one colonically absorbable form of levodopa through the gastrointestinal epithelia. Such absorption enhancers may, for example, open intercellular tight-junctions in the gastrointestinal tract or can modify the effect of cellular components, such as p-glycoprotein and the like. Suitable absorption enhancers include alkali metal salts of salicylic acid, such as sodium salicylate, caprylic or capric acid, such as sodium caprylate or sodium caprate, and the like. Suitable absorption enhancers also can include, for example, bile salts, such as sodium deoxycholate. Various p-glycoprotein modulators are described in Fukazawa et al., U.S. Pat. No. 5,112,817 and Pfister et al., U.S. Pat. No. 5,643,909. Various absorption enhancing compounds and materials are described, for example, in Burnside et al., U.S. Pat. No. 5,824,638, and Meezam et al., U.S. Application Publication No. 2006/0046962. Other adjuvants that enhance permeability of cellular membranes include resorcinol, surfactants, polyethylene glycol, and bile acids.

In certain embodiments, pharmaceutical compositions include an adjuvant that reduces enzymatic degradation of the at least one antipsychotic agent and/or at least one colonically absorbable form of levodopa. Microencapsulation using protenoid microspheres, liposomes, or polysaccharides may also be effective in reducing enzymatic degradation of administered compounds.

Pharmaceutical compositions may also include one or more pharmaceutically acceptable vehicles, including excipients, adjuvants, carriers, diluents, binders, lubricants, disintegrants, colorants, stabilizers, surfactants, fillers, buffers, thickeners, emulsifiers, wetting agents, and the like. Vehicles may be selected, for example, to alter the porosity and permeability of a pharmaceutical composition, alter hydration and disintegration properties, control hydration, enhance manufacturability, and the like.

In certain embodiments, pharmaceutical compositions are formulated for oral administration. Pharmaceutical compositions formulated for oral administration may provide for uptake of an antipsychotic agent and/or colonically absorbable form of levodopa throughout the gastrointestinal tract, or even in a particular region or regions of the gastrointestinal tract. In certain embodiments, pharmaceutical compositions may be formulated to enhance uptake of an antipsychotic agent and/or colonically absorbable form of levodopa from the lower gastrointestinal tract, and in certain embodiments, from the colon.

In certain embodiments, pharmaceutical compositions may further comprise a substance to enhance, modulate and/or control release, bioavailability, therapeutic efficacy, therapeutic potency, stability, etc. of the at least one antipsychotic agent and/or at least one colonically absorbable form of levodopa. For example, to enhance therapeutic efficacy, an antipsychotic agent and/or a colonically absorbable form of levodopa may be co-administered with one or more active agents to increase the absorption or diffusion of the drug from the gastrointestinal tract, or to inhibit degradation of the drug in the systemic circulation. In certain embodiments, an antipsychotic agent and/or colonically absorbable form of levodopa may be co-administered with an active agent having pharmacological effects that enhance the therapeutic efficacy of the antipsychotic agent, form of levodopa, and/or dopamine.

Pharmaceutical compositions comprising a colonically absorbable form of levodopa may take a form suitable for oral administration such as solutions, suspensions, emulsions, tablets, pills, pellets, capsules, capsules containing liquids, powders, sustained-release formulations, and suppositories.

Orally administered pharmaceutical compositions may comprise one or more optional agents, for example, sweetening agents such as fructose, aspartame, and saccharin; flavoring agents such as peppermint, oil of wintergreen, and cherry; coloring agents; and preserving agents, to provide a pharmaceutically palatable preparation. When in tablet or pill form, the compositions may be coated to delay disintegration and absorption in the gastrointestinal tract, thereby providing a sustained action over an extended period of time. Oral pharmaceutical compositions may include standard vehicles such as mannitol, lactose, starch, magnesium stearate, sodium saccharine, cellulose, magnesium carbonate, etc. Such vehicles may be of pharmaceutical grade.

For oral liquid preparations such as, for example, suspensions, elixirs, and solutions, suitable carriers, excipients or diluents include water, saline, alkyleneglycols (e.g., propylene glycol), polyalkylene glycols (e.g., polyethylene glycol), oils, alcohols, slightly acidic buffers having a pH ranging from about pH 4 to about pH 6 (e.g., acetate, citrate, ascorbate at between about 5 mM to about 50 mM), etc. Additionally, flavoring agents, preservatives, coloring agents, bile salts, acylcarnitines, and the like may be added.

Pharmaceutical compositions comprising at least one antipsychotic agent and at least one colonically absorbable form of levodopa may be formulated so as to provide immediate, sustained, or delayed release of the at least one antipsychotic agent after administration to the patient by employing procedures known in the art (see, e.g., Allen et al., “Ansel's Pharmaceutical Dosage Forms and Drug Delivery Systems,” 8th ed., Lippincott, Williams & Wilkins, August 2004).

In certain embodiments, pharmaceutical compositions provided by the present disclosure comprise at least one antipsychotic agent and at least one colonically absorbable form of levodopa. In certain embodiments, the at least one antipsychotic agent and the at least one colonically absorbable form of levodopa may be contained in separate pharmaceutical compositions. In certain embodiments, pharmaceutical compositions provided by the present disclosure comprise an amount of at least one antipsychotic agent that is effective for treating at least one positive symptom of schizophrenia. In certain embodiments, pharmaceutical compositions provided by the present disclosure comprise an amount of at least one colonically absorbable form of levodopa that is effective for treating at least one negative symptom of schizophrenia and/or at least one cognitive symptom of schizophrenia and that does not exacerbate or induce a positive symptom of schizophrenia.

Pharmaceutical compositions provided by the present disclosure that comprise at least one colonically absorbable form of levodopa, with or without at least one antipsychotic agent, may be formulated for administration in any manner that facilitates delivery of the form of levodopa to the large intestine, and in particular the colon. Formulations that facilitate delivery to the large intestine and the colon include, for example, intraperitoneal formulations, intracolonic formulations, intragastric formulations, and oral formulations.

Pharmaceutical compositions provided by the present disclosure that comprise at least one antipsychotic agent without at least one colonically absorbable form of levodopa may be formulated for any appropriate route of administration.

In embodiments in which at least one antipsychotic agent and at least one colonically absorbable form of levodopa are administered separately, the pharmaceutical compositions separately comprising each active agent may be provided in a kit form. Kits may include two separate pharmaceutical compositions—a first pharmaceutical composition comprising at least one antipsychotic agent and a second pharmaceutical composition comprising at least one colonically absorbable form of levodopa. Kits may include a container for containing the separate compositions such as a divided bottle or a divided foil packet. Kits may also include directions for administering the separate compositions. Kits may be particularly advantageous when the separate compositions are administered in different dosage forms, e.g., oral and parenteral, are administered at different dosage intervals, or when titration of the at least one antipsychotic agent and the at least one colonically absorbable form of levodopa is desired by the prescribing physician.

### Dosage Forms

Pharmaceutical compositions provided by the present disclosure may be formulated in unit dosage forms. Unit dosage form refers to a physically discrete unit suitable as a unitary dose for patients undergoing treatment, with each unit containing a predetermined quantity of at least one antipsychotic agent, at least one colonically absorbable form of levodopa, or both, calculated to produce the intended therapeutic effect. Unit dosage forms may be for a single daily dose, for a twice daily dose, or as one of multiple daily doses, e.g., 2, 3 or 4 times per day. When multiple daily doses are used, unit dosage forms may comprise the same or different amount of active agent(s) for each dose. One or more dosage forms can comprise a dose, which may be administered to a patient at a single point in time or during a time interval. Dosing may continue as long as required for effective treatment of the positive and the negative or cognitive symptoms of schizophrenia.

Controlled drug delivery systems may be designed to deliver a drug in such a way that a therapeutically effective and safe concentration of the drug is maintained in a relevant biological fluid, tissue, and/or organ for a continuous period of time. Controlled drug delivery may produce substantially constant plasma and/or blood levels of a drug as compared to fluctuations observed with immediate release dosage forms. For some drugs, maintaining a constant plasma, blood, and/or tissue concentration throughout the course of therapy is the most desirable mode of treatment. Immediate release of these drugs may cause blood levels to peak above the level required to elicit the desired response, which wastes the drug and may cause or exacerbate toxic or undesirable side effects. Controlled drug delivery may result in optimum therapy, and may reduce the frequency of dosing as well as reduce the severity of side effects. Examples of controlled release dosage forms include dissolution controlled systems, diffusion controlled systems, ion exchange resins, osmotically controlled systems, erodable matrix systems, pH independent formulations, gastric retention systems, and the like.

In certain embodiments, oral dosage forms provided by the present disclosure may be a controlled release dosage forms. Controlled delivery technologies may improve the absorption of the drug in a particular region or regions of the gastrointestinal tract. Oral controlled delivery technologies are particularly appropriate for use in administering colonically absorbable forms of levodopa.

The appropriate oral dosage form for a particular pharmaceutical composition may depend, at least in part, on the gastrointestinal absorption properties of the antipsychotic agent and/or colonically absorbable form of levodopa, the stability of the antipsychotic agent and/or colonically absorbable form of levodopa in the gastrointestinal tract, the pharmacokinetics of the antipsychotic agent and/or colonically absorbable form of levodopa, and the intended therapeutic profile. An appropriate controlled release oral dosage form may be selected for particular antipsychotic agents and/or colonically absorbable forms of levodopa. For example, gastric retention oral dosage forms may be appropriate for compounds absorbed primarily from the upper gastrointestinal tract, and sustained release oral dosage forms may be appropriate for compounds absorbed primarily from the lower gastrointestinal tract, including the colon.

Certain compounds are absorbed primarily from the small intestine. In general, compounds traverse the length of the small intestine in about 3 to about 5 hours. For compounds that are not easily absorbed by the small intestine or that do not dissolve readily, the window for active agent absorption in the small intestine may be too short to provide the desired therapeutic effect.

Gastric retention dosage forms, i.e., dosage forms that are retained in the stomach for a prolonged period of time, may increase the bioavailability of drugs that are most readily absorbed by the upper gastrointestinal tract. The residence time of a conventional dosage form in the stomach ranges from about 1 to about 3 hours. After transiting the stomach, there is approximately a about 3 to about 5 hour window of bioavailability before the dosage form reaches the colon. However, if the dosage form is retained in the stomach, the drug may be released before it reaches the small intestine and will enter the intestine in solution in a state in which it can be more readily absorbed. Another use of gastric retention dosage forms is to improve the bioavailability of drugs that are unstable to the basic conditions of the intestine (see, e.g., Hwang et al., Critical Reviews in Therapeutic Drug Carrier Systems, 1998, 15, 243-284).

To enhance drug absorption from the upper gastrointestinal tract, several gastric retention dosage forms have been developed. Examples include, hydrogels (see, e.g., Gutierrez-Rocca et al., U.S. Application Publication No. 2003/0008007), buoyant matrices (see, e.g., Lohray et al., U.S. Application Publication No. 2006/0013876), polymer sheets (see, e.g., Mohammad, U.S. Application Publication No. 2005/0249798), microcellular foams (see, e.g., Clarke et al., U.S. Application Publication No. 2005/0202090), and swellable dosage forms (see, e.g., Edgren et al., U.S. Application Publication No. 2005/0019409; Edgren et al., U.S. Pat. No. 6,797,283; Jacob et al., U.S. Application Publication No. 2006/0045865; Ayres, U.S. Application Publication No. 2004/0219186; Gusler et al., U.S. Pat. No. 6,723,340; Flashner-Barak et al., U.S. Pat. No. 6,476,006; Wong et al., U.S. Pat. Nos. 6,120,803 and 6,548,083; Shell et al., U.S. Pat. No. 6,635,280; and Conte et al., U.S. Pat. No. 5,780,057).

In swelling and expanding systems, dosage forms that swell and change density in relation to the surrounding gastric content may be retained in the stomach for longer than a conventional dosage form. Dosage forms may absorb water and swell to form a gelatinous outside surface and float on the surface of gastric content surface while maintaining integrity before releasing a drug. Fatty materials may be added to impede wetting and enhance flotation, for example, when hydration and swelling alone are insufficient. Materials that release gases may also be incorporated to reduce the density of the gastric retention dosage forms. Swelling may also significantly increase the size of a dosage form and thereby impede discharge of the non-disintegrated swollen solid dosage form through the pylorus into the small intestine. Swellable dosage forms may be formed by encapsulating a core containing drug and a swelling agent, or by combining a drug, swelling agent, and one or more erodible polymers.

Gastric retention dosage forms may also be in the form of a folded thin sheet containing a drug and water-insoluble diffusible polymer that opens in the stomach to its original size and shape, which is sufficiently large to prevent or inhibit passage of the expanded dosage form through the pyloric sphincter.

Floating and buoyancy gastric retention dosage forms may be designed to trap gases within sealed encapsulated cores that may float on the gastric contents, and thereby retained in the stomach for a longer time, e.g., from about 9 to about 12 hours. Due to the buoyancy effect, these systems may provide a protective layer preventing the reflux of gastric content into the esophageal region and may also be used for controlled release devices. Floating systems may, for example, contain hollow cores containing drug coated with a protective membrane. The trapped air in the cores floats the dosage from on the gastric content until the soluble ingredients are released and the system collapses. In other floating systems, cores comprise drug and chemical substances capable of generating gases when activated. For example, coated cores, comprising carbonate and/or bicarbonate may generate carbon dioxide from a reaction with hydrochloric acid in the stomach or incorporated organic acid in the system. The gas generated by the reaction is retained to float the dosage form. The inflated dosage form later collapses and clears form the stomach when the generated gas permeates slowly through the protective coating.

Bioadhesive polymers may also be used to provide a vehicle for controlled delivery of drugs to a number of mucosal surfaces in addition to the gastric mucosa (see, e.g., Mathiowitz et al, U.S. Pat. No. 6,235,313; and Illum et al., U.S. Pat. No. 6,207,197). Bioadhesive systems may be formed by incorporation of a drug and other excipients within a bioadhesive polymer. On ingestion, the polymer hydrates and adheres to the mucus membrane of the gastrointestinal tract. Bioadhesive polymers may be selected that adhere to a desired region or regions of the gastrointestinal tract. Bioadhesive polymers may be selected to optimized delivery to targeted regions of the gastrointestinal tract including the stomach and small intestine. The mechanism of adhesion is thought to be through the formation of electrostatic and hydrogen bonding at the polymer-mucus boundary. Jacob et al., U.S. Application Publication Nos. 2006/0045865 and 2005/0064027 disclose bioadhesive delivery systems which are useful for drug delivery to both the upper and lower gastrointestinal tract.

Ion exchange resins have also been shown to prolong gastric retention, potentially by adhesion.

Polymer matrices have also been used to achieve controlled release of drugs over a prolonged period of time. Sustained or controlled release may be achieved by limiting the rate by which the surrounding gastric fluid diffuses through the matrix and reach the drug, dissolves the drug and diffuses out again with the dissolved drug, or by using a matrix that slowly erodes, continuously exposing fresh drug to the surrounding fluid. Disclosures of polymer matrices that function by these methods are found, for example, in Skinner, U.S. Pat. Nos. 6,210,710 and 6,217,903; Rencher et al., U.S. Pat. No. 5,451,409; Kim, U.S. Pat. No. 5,945,125; Kim, PCT International Publication No. WO 96/26718; Ayer et al., U.S. Pat. No. 4,915,952; Akhtar et al., U.S. Pat. No. 5,328,942; Fassihi et al., U.S. Pat. No. 5,783,212; Wong et al., U.S. Pat. No. 6,120,803; and Pillay et al., U.S. Pat. No. 6,090,411.

Other drug delivery devices that remain in the stomach for extended periods of time include, for example, hydrogel reservoirs comprising particles (Edgren et al., U.S. Pat. No. 4,871,548); swellable hydroxypropylmethylcellulose polymers (Edgren et al., U.S. Pat. No. 4,871,548); planar bioerodible polymers (Caldwell et al., U.S. Pat. No. 4,767,627); structures comprising a plurality of compressible retention arms (Curatolo et al., U.S. Pat. No. 5,443,843); hydrophilic water-swellable, cross-linked polymer particles (Shell, U.S. Pat. No. 5,007,790); and albumin-cross-linked polyvinylpyrrolidone hydrogels (Park et al., J. Controlled Release 1992, 19, 131-134).

In certain embodiments, pharmaceutical compositions provided by the present disclosure may be formulated into a number of different dosage forms, which may be adapted to provide sustained release of an antipsychotic agent and/or a colonically absorbable form of levodopa upon oral administration. Sustained release oral dosage forms include any oral dosage form that maintains therapeutic concentrations of a drug in a biological fluid such as the plasma, blood, cerebrospinal fluid, or in a tissue or organ for a prolonged time period. Sustained release oral dosage forms may be used to release drugs over a prolonged time period and are useful when it is desired that a drug or drug form be delivered to the lower gastrointestinal tract. Sustained release oral dosage forms include diffusion-controlled systems such as reservoir devices and matrix devices, dissolution-controlled systems, osmotic systems, and erosion-controlled systems. Sustained release oral dosage forms and methods of preparing the same are well known in the art (see, for example, “Remington's Pharmaceutical Sciences,” Lippincott, Williams & Wilkins, 21st edition, 2005, Chapters 46 and 47; Langer, Science 1990, 249, 1527-1533; and Rosoff, “Controlled Release of Drugs,” 1989, Chapter 2).

In diffusion-controlled systems, water-insoluble polymers control the flow of fluid and the subsequent egress of dissolved drug from the dosage form. Both diffusional and dissolution processes are involved in release of drug from the dosage form. In reservoir devices, cores comprising a drug is coated with a polymer, and in matrix systems, a drug is dispersed throughout a polymer matrix. Cellulose polymers such as ethylcellulose and cellulose acetate may be used in reservoir devices. Examples of polymers useful in matrix systems include, but are not limited to, methacrylates, acrylates, polyethylene, acrylic acid copolymers, polyvinylchloride, high molecular weight polyvinylalcohols, cellulose derivates, and fatty compounds such as fatty acids, glycerides, and carnauba wax.

In dissolution-controlled systems, the rate of dissolution of the drug is controlled by slowly soluble polymers or by microencapsulation. Once the coating is dissolved, the drug becomes available for dissolution. By varying the thickness and/or the composition of the coating or coatings, the rate of drug release from the dosage form may be controlled. In some dissolution-controlled systems, a fraction of the total dose may comprise an immediate-release component. Dissolution-controlled systems include encapsulated/reservoir dissolution systems and matrix dissolution systems. Encapsulated dissolution systems may be prepared by coating particles or granules of drug with slowly soluble polymers of different thickness or by microencapsulation. Examples of coating materials useful in dissolution-controlled systems include, but are not limited to, gelatin, carnauba wax, shellac, cellulose acetate phthalate, and cellulose acetate butyrate. Matrix dissolution devices may be prepared, for example, by compressing a drug with a slowly soluble polymer carrier into a tablet form.

The rate of release of drug from osmotic pump systems is determined by the inflow of fluid across a semipermeable membrane into a reservoir, which contains an osmotic agent. Drug may be mixed with the osmotic agent or may be located in a reservoir. The dosage form may comprise one or more small orifices from which dissolved drug is pumped at a rate determined by the rate of entrance of water due to osmotic pressure. As osmotic pressure within the dosage form increases, the drug is released through the orifice(s). The rate of release is constant and may be controlled within tight limits yielding relatively constant plasma and/or blood concentrations of the drug. Osmotic pump systems may provide a constant release of drug independent of the environment of the gastrointestinal tract. The rate of drug release may be modified by altering the osmotic agent and/or the sizes of the one or more orifices.

Release of drug from erosion-controlled systems may be determined by the erosion rate of a carrier matrix. Drug is dispersed throughout a polymeric carrier matrix and the rate of drug release depends on the erosion rate of the polymer. The drug-containing polymer may degrade from the bulk and/or from the surface of the dosage form.

Sustained release oral dosage forms may be in any appropriate form for oral administration, such as, for example, in the form of tablets, pills, or granules. Granules may be filled into capsules, compressed into tablets, or included in a liquid suspension. Sustained release oral dosage forms may additionally include an exterior coating to provide, for example, acid protection, ease of swallowing, flavor, identification, etc.

In certain embodiments, sustained release oral dosage forms comprise a therapeutically effective amount of an antipsychotic agent, a therapeutically effective amount of a colonically absorbable form of levodopa and a pharmaceutically acceptable vehicle. In certain embodiments, sustained release oral dosage forms comprise less than a therapeutically effective amount of an antipsychotic agent, and/or less than a therapeutically amount of a colonically absorbable form of levodopa. In such cases, multiple dosage forms may be administered to a patient to provide a therapeutically effective dose or dosing may occur at intervals sufficient to provide a therapeutically effective plasma concentration of an antipsychotic agent and levodopa over time. Thus, multiple sustained release oral dosage forms, each dosage form comprising less than a therapeutically effective amount of an antipsychotic agent and/or less than a therapeutically effective amount of a colonically absorbable form of levodopa, may be administered at a single time or over a period of time to provide a therapeutically effective dose or regimen for treating schizophrenia in a patient.

Sustained release oral dosage forms provided by the present disclosure may release the antipsychotic agent and/or colonically absorbable form of levodopa from the dosage form to facilitate the ability of the antipsychotic agent and/or colonically absorbable form of levodopa to be absorbed from an appropriate region of the gastrointestinal tract, such as for example, in the large intestine, and in particular the colon. In certain embodiments, sustained release oral dosage forms release the antipsychotic agent and/or the colonically absorbable form of levodopa from the dosage form over a period of at least about 4 hours, for example over at least about 8 hours, at least about 12 hours, at least about 16 hours, at least about 20 hours, at least about 24 hours, and in certain embodiments, more than about 24 hours. In certain embodiments, sustained release oral dosage forms release the antipsychotic agent and/or the colonically absorbable form of levodopa in a delivery pattern ranging from about 0 wt % to about 20 wt % in from about 0 to about 4 hours, from about 20 wt % to about 50 wt % in from about 0 to about 8 hours, from about 55 wt % to about 85 wt % in from about 0 to about 14 hours, and from about 80 wt % to about 100 wt % in from about 0 to about 24 hours. In certain embodiments, sustained release oral dosage forms release the antipsychotic agent and/or the colonically absorbable form of levodopa in a delivery pattern ranging from about 0 wt % to about 20 wt % in from about 0 to about 4 hours, from about 20 wt % to about 50 wt % in from about 0 to about 8 hours, from about 55 wt % to about 85 wt % in from about 0 to about 14 hours, and from about 80 wt % to about 100 wt % in from about 0 to about 20 hours. In certain embodiments, sustained release oral dosage forms release the antipsychotic agent and/or the colonically absorbable form of levodopa in a delivery pattern ranging from about 0 wt % to about 20 wt % in from about 0 to about 2 hours, from about 20 wt % to about 50 wt % in from about 0 to about 4 hours, from about 55 wt % to about 85 wt % in from about 0 to about 7 hours, and from about 80 wt % to about 100 wt % in from about 0 to about 8 hours.

Sustained release oral dosage forms comprising at least one antipsychotic agent and/or at least one colonically absorbable form of levodopa may provide a concentration of the antipsychotic agent and/or levodopa in the plasma, blood, or tissue of a patient over time, following oral administration to the patient. The concentration profile of the antipsychotic agent and/or levodopa may exhibit an AUC that is proportional to the dose of the antipsychotic agent and/or colonically absorbable form of levodopa administered to the patient.

Regardless of the type controlled release oral dosage form used, an antipsychotic agent and/or a colonically absorbable form of levodopa may be released from an orally administered dosage form over a sufficient period of time to provide prolonged therapeutic concentrations of the antipsychotic agent and/or levodopa in the plasma and/or blood of a patient. Following administration, dosage forms comprising a antipsychotic agent and/or a colonically absorbable form of levodopa may provide a therapeutically effective concentration the antipsychotic agent and/or levodopa in the plasma and/or blood of a patient during a continuous time period for at least about 4 hours, for example for at least about 8 hours, for at least about 12 hours, for at least about 16 hours, for at least about 20 hours, and in certain embodiments, for more than 24 hours following administration of the dosage form to the patient. The continuous period of time during which a therapeutically effective concentration of the antipsychotic agent and levodopa is maintained may be the same or different. The continuous period of time during which a therapeutically effective plasma concentration of the antipsychotic agent and/or levodopa is maintained may begin shortly after administration or after a time interval following administration.

In certain embodiments, oral dosage forms for treating schizophrenia in a patient comprise at least one antipsychotic agent and/or at least one colonically absorbable form of levodopa, wherein the oral dosage form is adapted to provide, after a single administration of the oral dosage form to the patient, a therapeutically effective concentration of the at least one antipsychotic agent and/or levodopa in the plasma of the patient during a first continuous time period chosen from at least about 4 hours, for example at least about 8 hours, at least about 12 hours, and at least about 16 hours, at least about 20 hours, or even at least about 24 hours.

In certain embodiments, dosage forms provided by the present disclosure may be designed using, for example, a combination of one of the above identified technologies or other technology known to those skilled in the art of the pharmaceutical sciences, to optimize the amount of the antipsychotic agent and/or the colonically absorbable form of levodopa absorbed from the gastrointestinal tract and which provides a plasma and/or blood concentration of the antipsychotic agent and/or levodopa within a therapeutically effective window for a continuous period of time. For example, dosage forms may provide for absorption of the antipsychotic agent and/or the colonically absorbable form of levodopa from both the small intestine and from the large intestine. In certain embodiments, the plasma and/or blood concentration of the antipsychotic agent and/or levodopa may achieve or approach zero-order kinetics for a continuous period of time after the dosage form is administered to a patient.

### Uses

Pharmaceutical compositions and dosage forms provided by the present disclosure may be used to treat a positive symptom of schizophrenia, a negative or cognitive symptom of schizophrenia, both a positive and a negative or cognitive symptom of schizophrenia and/or closely associated psychotic disorders such as schizophreniform disorder, schizoaffective disorder, delusional disorder, brief psychotic disorder, shared psychotic disorder, psychotic disorder due to a general medical condition, substance-induced psychotic disorder, and/or unspecified psychotic disorders in a patient (DSM-IV-TR, 4^{th} Edition, pp. 297-344, American Psychiatric Association, 2005). Positive symptoms of schizophrenia include delusion and hallucination. Negative symptoms of schizophrenia include affect blunting, anergia, alogia, and social withdrawal. Cognitive symptoms of schizophrenia include impairment in obtaining, organizing, and using intellectual knowledge. In certain embodiments, pharmaceutical compositions and dosage forms provided by the present disclosure may be used to treat both a positive and a negative or cognitive symptom of schizophrenia by orally administering the pharmaceutical composition or dosage form to a patient in need of such treatment.

In certain embodiments, an antipsychotic agent and a colonically absorbable form of levodopa may be used to treat both a positive and a negative or cognitive symptom of schizophrenia or a closely associated psychotic disorders such as schizophreniform disorder, schizoaffective disorder, delusional disorder, brief psychotic disorder, shared psychotic disorder, psychotic disorder due to a general medical condition, substance-induced psychotic disorder, and unspecified psychotic disorders in a patient. In certain embodiments, an antipsychotic agent and a colonically absorbable form of levodopa may used to treat a positive and a negative or cognitive symptom of schizophrenia by orally administering the compounds to a patient in need of such treatment. In certain embodiments, an antipsychotic agent may be administered to a patient by any appropriate route, such as orally, parenterally, or by bolus injection, and the colonically absorbable form of levodopa may be administered orally.

The efficacy of pharmaceutical compositions, dosage forms, and methods provided by the present disclosure for treating schizophrenia may be determined by methods known to those skilled in the art. For example, negative, positive, and/or cognitive symptom(s) of schizophrenia may be measured before and after treatment of the patient. Reduction in such symptom(s) indicates that a patient's condition has improved. Improvement in the symptoms of schizophrenia may be assessed using, for example, the Scale for Assessment of Negative Symptoms (SANS), Positive and Negative Symptoms Scale (PANSS) (see, e.g., Andreasen, 1983, Scales for the Assessment of Negative Symptoms (SANS), Iowa City, Iowa; and Kay et al., Schizophrenia Bulletin 1987, 13, 261-276), and using Cognitive Deficits tests such as the Wisconsin Card Sorting Test (WCST) and other measures of cognitive function (see, e.g., Keshavan et al., Schizophr Res 2004, 70(2-3), 187-194; Rush, Handbook of Psychiatric Measures, American Psychiatric Publishing 2000; Sajatovic and Ramirez, Rating Scales in Mental Health, 2nd ed, Lexi-Comp, 2003, Keefe, et al., Schizophr Res. 2004, 68(2-3), 283-97; and Keefe et al., Neuropsychopharmacology, advance online publication 19 Apr. 2006, doi:10.1038/sj.npp.1301072).

The efficacy of pharmaceutical compositions, dosage forms, and methods provided by the present disclosure may be evaluated using animal models of schizophrenic disorders (see e.g., Geyer and Moghaddam, in “Neuropsychopharmacology,” Davis et al., Ed., Chapter 50, 689-701, American College of Neuropsychopharmacology, 2002). For example, conditioned avoidance response behavior (CAR) and catalepsy tests in rats are shown to be useful in predicting antipsychotic activity and EPS effect liability, respectively (Wadenberg et al., Neuropsychopharmacology, 2001, 25, 633-641).

In certain embodiments, methods of treating schizophrenia in a patient comprise orally administering to a patient in need of such treatment a pharmaceutical composition comprising at least one antipsychotic agent and at least one colonically absorbable form of levodopa. In certain embodiments, methods of treating schizophrenia in a patient comprise orally administering to a patient in need of such treatment a dosage form comprising at least one antipsychotic agent and at least one colonically absorbable form of levodopa. In certain embodiments, methods of treating schizophrenia in a patient comprise orally administering to a patient in need of such treatment at least one antipsychotic agent in an amount that is effective for treating a positive symptom of schizophrenia in the patient, and at least one colonically absorbable form of levodopa in an amount that is effective for treating a negative or cognitive symptom of schizophrenia in the patient and that does not exacerbate or induce a positive symptom of schizophrenia in the patient. In certain embodiments, methods of treating schizophrenia in a patient comprise administering to a patient in need of such treatment at least one antipsychotic agent in an amount that is effective for treating a positive symptom of schizophrenia in the patient, and orally administering to a patient in need of such treatment, at least one colonically absorbable form of levodopa in an amount that is effective for treating a negative or cognitive symptom of schizophrenia in the patient and that does not exacerbate or induce a positive symptom of schizophrenia in the patient.

### Doses

An antipsychotic agent and a colonically absorbable form of levodopa may be used to treat both a positive and a negative or cognitive symptom of schizophrenia.

The amount of an antipsychotic agent and a colonically absorbable form of levodopa that will be effective in the treatment of schizophrenia will depend on the nature and severity of the schizophrenia, and may be determined by standard clinical techniques known in the art. In addition, in vitro or in vivo assays may be employed to help identify optimal dosage ranges. For systemic administration, a therapeutically effective dose may be estimated initially from in vitro assays. For example, a dose may be formulated in animal models to achieve a beneficial circulating composition concentration range. Initial doses can also be estimated from in vivo data, e.g., animal models, using techniques that are known in the art. Such information may be used to more accurately determine useful doses in humans. One having ordinary skill in the art may optimize administration to humans based on animal data. The amount of a compound administered that will be effective in treating schizophrenia may depend on, among other factors, the patient being treated, the weight of the patient, the health of the patient, the disease being treated, the severity of the affliction, the route of administration, the potency of the compound, and the judgment of the prescribing physician.

A dose may be administered in a single dosage form or in multiple dosage forms. When multiple dosage forms are used, the amount of compound contained within each dosage form may be the same or different. The amount of an antipsychotic agent and/or colonically absorbable form of levodopa contained in a dose may depend on the route of administration and whether the disease, disorder, or condition in a patient is effectively treated by acute, chronic, or a combination of acute and chronic administration. The treatment of schizophrenia is typically accomplished by chronic administration.

An administered dose is less than a toxic dose. Toxicity of compounds and pharmaceutical compositions provided by the present disclosure may be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. In certain embodiments, a pharmaceutical composition may exhibit a high therapeutic index. The data obtained from these cell culture assays and animal studies may be used in formulating a dosage range that is not toxic for use in humans. Doses of compounds or pharmaceutical compositions provided by the present disclosure may be within a range that provides a circulating concentration of the compound or metabolite thereof in for example the blood, plasma, cerebrospinal fluid, or central nervous system, that includes the effective dose and that exhibits little or no toxicity. Doses of compounds or pharmaceutical compositions provided by the present disclosure may also be within a range that provides a therapeutically effective concentration of the compound or metabolite thereof in the target tissue or organ of a patient and that exhibits little or no toxicity or an unacceptable side effect profile. For example, in certain embodiments, doses of a colonically absorbable form of levodopa will be within a range that provides a therapeutically effective plasma levodopa concentration and a therapeutically effective concentration of dopamine in the prefrontal cortex, and/or that exhibits little or no toxicity and/or an unacceptable side effect profile, such as that administering the colonically absorbable form of levodopa does not exacerbate or induce a positive symptom of schizophrenia. Doses may vary within this range depending upon the dosage form employed and the route of administration utilized.

During treatment, doses and dosing schedules may provide sufficient or steady state levels of an effective amount of an antipsychotic agent to treat a positive symptom of schizophrenia and an effective amount of a colonically absorbable form of levodopa to treat a negative or cognitive symptom of schizophrenia without exacerbating or inducing a positive symptom of schizophrenia, which may be the same positive symptom being treated with the antipsychotic agent or a different positive symptom than that being treated. Doses and dosing schedules of one or both of the active compounds, i.e., the antipsychotic agent and colonically absorbable form of levodopa, may be modified as necessary during the course of the treatment.

Because the use of levodopa in schizophrenia therapy can have both positive and negative effects, the ability to control the plasma levodopa concentration within a therapeutically effective window may be an important factor in the efficacy of a combined treatment approach. The dopamine hypothesis of schizophrenia postulates that there is an excessively high concentration of dopamine in the striatum that causes or is associated with the positive symptoms of schizophrenia, and a depletion of dopamine in the prefrontal cortex, which is associated with the negative or cognitive symptoms of schizophrenia. Based on this model of schizophrenia, an appropriate therapeutically effective circulating plasma levodopa concentration for treating schizophrenia would augment a depleted dopamine concentration in the prefrontal cortex, and at the same time would not be effective in overcoming the effects of the antipsychotic agent used to treat the positive symptoms of schizophrenia. A therapeutically effective circulating plasma antipsychotic agent concentration for treating schizophrenia will be effective in treating at least one positive symptom of schizophrenia without exacerbating or inducing undesirable side effects, such as extrapyramidal side (EPS) effects.

Doses of a colonically absorbable form of levodopa administered to a patient may be in an amount that is effective in treating a negative or cognitive symptom of schizophrenia, and that does not exacerbate or induce a positive symptom of schizophrenia. Studies implicate the role of dysregulation of dopamine synthesis in the prefrontal cortex in schizophrenia and postulate that supplementation of the low dopamine levels in the prefrontal cortex can specifically address cognitive impairment associated with schizophrenia (Mohr et al., J Psychiatric Res 2005, 39, 241-250; and Lindstrom et al., Biol Psychiatry 1999, 46, 681-688). Accordingly, doses of a colonically absorbable form of levodopa capable of treating a negative or cognitive symptom of levodopa may augment the concentration of dopamine in the prefrontal cortex. Clinical studies on the efficacy of schizophrenia therapy using a combination of an antipsychotic agent such as a typical antipsychotic and levodopa suggest that daily dosages ranging from about 300 mg to about 1,200 mg of levodopa is effective in ameliorating the positive as well as the negative and/or cognitive symptoms of schizophrenia and results in improved therapy compared to administering the antipsychotic drug alone (see, e.g., Kay and Opler, Int'l J Psychiatry in Medicine 1985, 15(3), 293-298; Inanaga et al., Folia Psychiatrica et Neurologica Japonica 1975, 29(2), 123-143; Gerlach et al., Psychopharmacologia 1975, 44, 105-110; Jaskiw and Popli, Psychopharmacology 2004, 17(1), 365-374; Buchanan et al., Aust N Z J Psychiatry 1975, 9(4), 269-71; Gerlach and Luhdorf, Psychopharmacologiea 1975, 44(1), 105-10; and Inanaga et al., Folia Psychiatr Neurol Jpn 1975, 29(3), 197-205).).

These studies investigating the adjunctive treatment of schizophrenia were generally performed using orally administered formulations of levodopa and a decarboxylase inhibitor. For example, Sinemet®, an immediate release form of levodopa and carbidopa (100 mg: 25 mg) provides a maximum plasma levodopa concentration of about 3,256 ng/mL, at about 0.5 hours after dosing, and a minimum plasma levodopa concentration of about 74 ng/mL, at about 8 hours after oral administration. The bioavailability of levodopa from Sinemet® is about 99% relative to the bioavailability of intravenously administered levodopa in healthy elderly patients (Merck & Co., Inc, product literature 2002). Sinemet® CR, a controlled release gastric retentive dosage form of levodopa and carbidopa (200 mg: 50 mg), provides a levodopa bioavailability of about 70-75% relative to intravenously administered levodopa or standard Sinemet® in elderly patients. A single dose of Sinemet® CR provides a maximum plasma levodopa concentration of about 1,151 ng/mL at about 2 hours after oral administration, with a minimum levodopa concentration of about 163 ng/mL at about 8 hours following administration. The mean plasma levodopa concentration during an 8-hour time period following oral administration of either an immediate release or controlled release Sinemet® dosage form ranges from about 200 ng/mL to about 400 ng/mL (see, e.g., Sinemet® product literature, Merck & Co., Inc.; Yeh et al., Neurology 1989, 39(Suppl 2), S25-S38). Thus, Sinemet® dosage forms provide a wide range of plasma levodopa concentrations during a snort, about 8-hour, time period. Such a wide fluctuation in the circulating levodopa plasma concentration is not expected to be effective, or as effective, in treating the negative and/or cognitive symptoms of schizophrenia where studies suggest that relatively tight control of the dopamine concentration in the prefrontal cortex is necessary to treat the negative and/or cognitive symptoms of schizophrenia without simultaneously exacerbating or inducing the positive symptoms of schizophrenia.

Thus, in certain embodiments, doses of a colonically absorbable form of levodopa for treating a negative and/or cognitive symptom of schizophrenia in a patient may range from about 100 mg-equivalents/day of levodopa to about 1,000 mg-equivalents/day of levodopa. In certain embodiments, doses of a colonically absorbable form of levodopa for treating a negative and/or cognitive symptom of schizophrenia in a patient may range from about 100 mg-equivalents/day of levodopa to about 800 mg-equivalents/day of levodopa. In certain embodiments, doses of a colonically absorbable form of levodopa for treating a negative and/or cognitive schizophrenia in a patient may range from about 200 mg-equivalents/day of levodopa to about 500 mg-equivalents/day of levodopa. For comparison, a dose of about 1,000 mg/day of levodopa in combination with about 200 mg/day of carbidopa is recommended for use in treating Parkinson's disease (Sinemet® and Sinemet® CR product literature, Merck & Co., Inc.). A daily dose may be administered in 3 doses per day, and for lesser daily doses, 2 or three times per day with each dose comprising from about 200 mg to about 300 mg of levodopa.

In certain embodiments, pharmaceutical compositions or oral dosage forms comprising a colonically absorbable form of levodopa for treating schizophrenia in a patient may provide a plasma levodopa concentration from about 50 ng/mL to about 1,000 ng/mL during a continuous period of time chosen from at least about 4 hours, at least about 8 hours, at least about 16 hours, and at least about 24 hours following oral administration of the pharmaceutical composition or oral dosage form the patient. In certain embodiments, pharmaceutical compositions or oral dosage forms comprising a colonically absorbable form of levodopa for treating schizophrenia in a patient may provide a plasma levodopa concentration from about 100 ng/mL to about 800 ng/mL during a continuous period of time chosen from at least about 4 hours, at least about 8 hours, at least about 16 hours, and at least about 24 hours following oral administration of the pharmaceutical composition or oral dosage form the patient. In certain embodiments, pharmaceutical compositions or oral dosage forms comprising a colonically absorbable form of levodopa for treating schizophrenia in a patient may provide a plasma levodopa concentration from about 500 ng/mL to about 1,000 ng/mL during a continuous period of time chosen from at least about 4 hours, at least about 8 hours, at least about 16 hours, and at least about 24 hours following oral administration of the pharmaceutical composition or oral dosage form the patient.

In certain embodiments, pharmaceutical compositions or oral dosage forms comprising a colonically absorbable form of levodopa for treating schizophrenia in a patient may provide a mean plasma levodopa concentration from about 50 ng/mL to about 500 ng/mL during a continuous period of time chosen from at least about 4 hours, at least about 8 hours, at least about 16 hours, and at least about 24 hours following oral administration of the pharmaceutical composition or oral dosage form the patient. In certain embodiments, pharmaceutical compositions or oral dosage forms comprising a colonically absorbable form of levodopa for treating schizophrenia in a patient may provide a mean plasma levodopa concentration from about 100 ng/mL to about 400 ng/mL during a continuous period of time chosen from at least about 4 hours, at least about 8 hours, at least about 16 hours, and at least about 24 hours following oral administration of the pharmaceutical composition or oral dosage form to the patient.

In certain embodiments, pharmaceutical compositions or dosage forms comprising a colonically absorbable form of levodopa further comprise an amount of a decarboxylase inhibitor such as carbidopa or benserazide that is from about 5 wt % to about 30 wt % of the amount of levodopa, and in certain embodiments, from about 10 wt % to about 20 wt % of the amount of levodopa. In certain embodiments, pharmaceutical compositions or dosage forms comprising a colonically absorbable form of levodopa comprise an amount of a catechol-O-methyltransferase inhibitor such as entacapone or tolcapone that is from about 5 wt % to about 30 wt % of the about of levodopa, and in certain embodiments, from about 10 wt % to about 20 wt % of the amount of levodopa.

Certain embodiments of the pharmaceutical compositions and dosage forms provided by the present disclosure provide a concentration of levodopa in the plasma of a patient, which is effective for treating a negative or cognitive symptom of schizophrenia and does not exacerbate a positive symptom of schizophrenia for a prolonged period of time. Thus, the plasma levodopa concentration may be maintained within a therapeutically effective window which is less than the plasma levodopa concentration that produces an exacerbation of a positive symptom of schizophrenia, and greater than the plasma levodopa concentration that is effective in treating a negative or cognitive symptom of schizophrenia. The bounds of the therapeutically effective window may depend, for example, on the severity of the positive, negative, or cognitive symptoms, the nature of the positive, negative, or cognitive symptoms, the health of the patient, and the like. In certain embodiments, the plasma levodopa concentration never exceeds that which exacerbates a positive symptom of schizophrenia, or does not exceed a concentration that exacerbates a positive symptom of schizophrenia for a sufficiently long period of time to result in a significantly reduced efficacy of the treatment of the positive symptom of schizophrenia.

A dose of an antipsychotic agent administered to a patient may be an amount that is effective in treating a positive symptom of schizophrenia. A dose of an antipsychotic agent for treating a positive symptom of schizophrenia may range from about 0.25 mg/day to about 5,000 mg/day, in certain embodiments from about 2 mg/day to about 1,000 mg/day, and in certain embodiments from about 10 mg/day to about 300 mg/day. The effective dosage may depend, for example, on the route of administration, the age of the patient, and the specific antipsychotic agent administered. Examples of daily doses for certain typical antipsychotics are:

acetophenazine (about 10-2,000 mg/day, and in certain embodiments, about 30-500 mg/day);

chlorpromazine (about 5-2000 mg/day, for example, about 30-800 mg/day, and in certain embodiments, about 20-300 mg/day);

chlorprothixene (about 5-2,000 mg/day, for example, about 30-500 mg/day, and in certain embodiments, about 75-200 mg/day);

droperidol (about 0.25-500 mg/day, for example, about 1-100 mg/day, and in certain embodiments, 0.5-20 mg/day);

fluphenazine (about 0.25-200 mg/day, for example, about 0.5-40 mg/day, and in certain embodiments, about 0.25-20 mg/day);

haloperidol (about 0.5-500 mg/day, for example, about 1-100 mg/day);

loxapine (about 1-1000 mg/day, for example, about 10-250 mg/day, and in certain embodiments, about 70-200 mg/day);

mesoridazine (about 1-1,000 mg/day, for example about 30-400 mg/day, and in certain embodiments, about 30-150 mg/day);

methotrimeprazine (about 10-200 mg/day, for example, about 50-75 mg/day);

molindone (1-1,000 mg/day, for example, about 15-225 mg/day);

perphenazine (about 0.5-300 mg/day, for example, about 10-70 mg/day);

pimozide (about 0.2-500 mg/day, for example, about 0.5-10 mg/day);

thiothixene (about 1-200 mg/day, for example, about 6-60 mg/day);

trifluoperazine (about 0.5-200 mg/day, for example, about 2-40 mg/day);

thioridazine (about 5-2,000 mg/day, for example, about 20-800 mg/day, and in certain embodiments, about 50-800 mg/day); and

triflupromazine (about 10-300 mg/day, for example, about 60-150 mg/day).

Examples of daily doses for certain atypical antipsychotics are:

clozapine (about 5-2000 mg/day, for example, about 12-900 mg/day);

olanzapine (about 1-100 mg/day, for example, about 5-10 mg/day);

quetiapine (about 1-2000 mg/day, for example, about 50-750 mg/day); and

risperidone (about 0.25-500 mg/day, for example, about 2-16 mg/day).

Examples of daily doses for other antipsychotics agents are:

butaperazine (about 0.5-500 mg/day, for example, about 1-200 mg/day);

carphenazine, (about 0.5-3000 mg/day, for example, about 1-1000 mg/day);

piperacetazine (about 0.5-500 mg/day, for example, about 1-2000 mg/day);

remoxipride (about 0.5-5000 mg/day, for example, about 1-2000 mg/day);

sulpiride (about 0.5-5000-mg/day, for example, about 1-2000 mg/day); and

ziprasidone (about 0.5-500 mg/day, for example, about 1-200 mg/day).

An antipsychotic agent and a colonically absorbable form of levodopa can act synergistically to treat symptoms of schizophrenia. For example, the antipsychotic agent can treat a positive symptom of schizophrenia, and the colonically absorbable form of levodopa can treat a negative or cognitive symptom of schizophrenia, thereby resulting in an overall improved therapy for schizophrenia.

### Administration

An antipsychotic agent and a colonically absorbable form of levodopa or a pharmaceutical composition thereof can be administered to a patient suffering from schizophrenia in less than one dose per day, one dose per day, or in more than one dose per day, e.g., 2, 3 or 4 doses. A patient can be treated for at least about one week, for at least several weeks, e.g., at least about 4, about 6, or about 8 weeks, for several months, e.g., at least about 4, about 8, or about 12 months, or for several years. If necessary, the treatment can continue as long as necessary to maintain the patient's symptoms under control throughout his or her life.

In certain embodiments, pharmaceutical dosage forms comprising at least one colonically absorbable form of levodopa and optionally at least one antipsychotic agent may be administered for delivery into the large intestine, including the colon. Routes of administration for delivery into the large intestine include, for example, enteral, intra-abdominal, intragastric, and oral. In certain embodiments, pharmaceutical dosage forms comprising at least one colonically absorbable form of levodopa and optionally at least one antipsychotic agent may be administered orally.

An antipsychotic agent and a colonically absorbable form of levodopa may be co-administered simultaneously or sequentially in any order, or as a single pharmaceutical composition. An antipsychotic agent and a colonically absorbable form of levodopa may be administered by the same or by a different route. For example, a colonically absorbable form of levodopa may be administered orally and the antipsychotic agent may be administered orally, intravenously, or by depot injection.

### Combination Therapy

The pharmaceutical compositions provided by the present disclosure may comprise, in addition to an antipsychotic agent and a colonically absorbable form of levodopa, one or more additional therapeutic agents effective for treating schizophrenia or a different disease, disorder, or condition other than schizophrenia. In certain embodiments, methods provided by the present disclosure include administration of an antipsychotic agent and a colonically absorbable form of levodopa and one or more other therapeutic agents provided that the combined administration does not inhibit the therapeutic efficacy of the one or more antipsychotic agents and a colonically absorbable form of levodopa and/or does not produce adverse combination effects.

In certain embodiments, an antipsychotic agent and a colonically absorbable form of levodopa may be administered concurrently with the administration of another therapeutic agent, such as for example, a compound for treating a positive symptom of schizophrenia, a negative symptom of schizophrenia, and/or a cognitive symptom of schizophrenia. In some embodiments, an antipsychotic agent and a colonically absorbable form of levodopa may be administered prior or subsequent to administration of another therapeutic agent, such as for example, a compound for treating a positive, negative, and/or cognitive symptom of schizophrenia.

In certain embodiments, an antipsychotic agent and/or a colonically absorbable form of levodopa may be administered concurrently with the administration of another therapeutic agent, which may be part of the same pharmaceutical composition or dosage form as, or in a different composition or dosage form from, that containing the antipsychotic agent and/or the colonically absorbable form of levodopa. In certain embodiments, an antipsychotic agent and a colonically absorbable form of levodopa may be administered prior or subsequent to administration of another therapeutic agent. In certain embodiments of combination therapy, the combination therapy comprises alternating between administering the antipsychotic agent and the colonically absorbable form of levodopa and a composition comprising another therapeutic agent, e.g. to minimize adverse side effects associated with a particular drug. When an antipsychotic agent and a colonically absorbable form of levodopa is administered concurrently with another therapeutic agent that potentially may produce adverse side effects including, but not limited to, toxicity, the therapeutic agent may be administered at a dose that falls below the threshold at which the adverse side effect is elicited.

In certain embodiments, an antipsychotic agent and a colonically absorbable form of levodopa, or pharmaceutical compositions thereof may be administered to a patient for the treatment of schizophrenia in combination with a therapy or treatment known or believed to be effective in the treatment schizophrenia or a symptom of schizophrenia.

For example, in certain embodiments, an antipsychotic agent and a colonically absorbable form of levodopa, or pharmaceutical compositions thereof may be administered to a patient for the treatment of schizophrenia in conjunction with a social therapy for treating schizophrenia such as rehabilitation, community support activities, cognitive behavioral therapy, training in illness management skills, participation in self-help groups, and/or psychotherapy. Examples of psychotherapy useful for treating schizophrenia include Alderian therapy, behavior therapy, existential therapy, Gestalt therapy, person-centered therapy, psychoanalytic therapy, rational-emotive and cognitive-behavioral therapy, reality therapy, and transactional analysis.

In certain embodiments, pharmaceutical compositions provided by the present disclosure may be co-administered with another drug useful for treating a symptom of schizophrenia or a disease, disorder, or condition associated with schizophrenia and that is not an antipsychotic agent. For example, pharmaceutical compositions may be co-administered with an antidepressant, such as, but not limited to alprazolam, amitriptyline, amoxapine, bupropion, citalopram, clomipramine, desipramine, eoxepin, escitapopram, fluoxetine, fluvoxamine, imipramine, maprotiline, methylphenidate, mirtazapine, nefazodone, nortriptyline, paroxetine, phenelzine, protriptyline, sertraline, tranylcypromine, trazodone, trimipramine, venlafaxine, and combinations of any of the foregoing.

### EXAMPLES

The invention is further defined by reference to the following examples, which describe uses of compositions provided by the present disclosure. It will be apparent to those skilled in the art that many modifications, both to materials and methods, may be practiced without departing from the scope of the disclosure.

### Example 1

### Uptake of a Compound of Colonically Absorbable Forms of Levodopa in Rats

Sustained release oral dosage forms, which release drug slowly over periods of about 6 to about 24 hours, generally release a significant proportion of the dose within the colon. Thus, drugs suitable for use in such dosage forms should be colonically absorbed. This experiment was conducted to assess the uptake and resultant plasma/blood levels of levodopa, following intracolonic administration of a colonically absorbable form of levodopa with co-administration of carbidopa (intracolonically, intraperitoneally, or orally), and thereby determine the suitability of a colonically absorbable form of levodopa for use in an oral sustained release dosage form. Bioavailability of levodopa following co-administration of a colonically absorbable form of levodopa carbidopa was calculated relative to oral co-administration of levodopa and carbidopa.

Step A: Administration Protocol

Rats were obtained commercially and were pre-cannulated in the both the ascending colon and the jugular vein. Animals were conscious at the time of the experiment. All animals were fasted overnight and until 4 hours post-dosing of a colonically absorbable form of levodopa. Carbidopa was administered as a solution in water or citrate buffer either orally, intraperitoneally, or intracolonically at a dose equivalent to 25 mg of carbidopa per kg. Either at the same time or 1 hour after carbidopa dosing, a colonically absorbable form of levodopa was administered as a solution (in water) directly into the colon via the cannula at a dose equivalent to 75 mg of levodopa per kg. Blood samples (0.3 mL) were obtained from the jugular cannula at intervals over 8 hours and were immediately quenched with sodium metabisulfite to prevent oxidation of levodopa and levodopa prodrug. Blood was then further quenched with methanol/perchloric acid to prevent hydrolysis of the levodopa prodrug. Blood samples were analyzed as described below.

Step B: Sample Preparation for Colonically Absorbed Drug

Methanol/perchloric acid (300 μL) was added to blank 1.5 mL Eppendorf tubes. Rat blood (300 μL) was collected into EDTA tubes containing 75 μL of sodium metabisulfite at different times and vortexed to mix. A fixed volume of blood (100 μL) was immediately added into the Eppendorf tube and vortexed to mix. Ten μL of a levodopa standard stock solution (0.04, 0.2, 1, 5, 25, and 100 μg/mL) and 10 μL of the 10% sodium metabisulfite solution was added to 80 μL of blank rat blood to make up a final calibration standard (0.004, 0.02, 0.1, 0.5, 2.5, and 10 μg/mL). Methanol/perchloric acid (300 μL of 50/50) was then added into each tube followed by the addition of 20 μL of p-chlorophenylalanine. The samples were vortexed and centrifuged at 14,000 rpm for 10 min. The supernatant was analyzed by LC/MS/MS.

Step C: LC/MS/MS Analysis

An API 4000 LC/MS/MS spectrometer equipped with Agilent 1100 binary pumps and a CTC HTS-PAL autosampler were used in the analysis. A Zorbax XDB C8 4.6×150 mm column was used during the analysis. The mobile phases were (A) 0.1% formic acid, and (B) acetonitrile with 0.1% formic acid. The gradient condition was: 5% B for 0.5 min, then to 98% B in 3 min, then maintained at 98% B for 2.5 min. The mobile phase was then returned to 2% B for 2 min. A TurboIonSpray source was used on the API 4000. The analysis was done in positive ion mode and the MRM transition for each analyte was optimized using standard solution. Five μL of each sample was injected. Non-compartmental analysis was performed using WinNonlin software (v.3.1 Professional Version, Pharsight Corporation, Mountain View, Calif.) on individual animal profiles. Summary statistics on major parameter estimates was performed for Cₘₐₓ (peak observed concentration following dosing), Tₘₐₓ (time to maximum concentration is the time at which the peak concentration was observed), AUC₍₀₋ₜ₎ (area under the serum concentration-time curve from time zero to last collection time, estimated using the log-linear trapezoidal method), AUC_{(0-∞)} (area under the blood concentration time curve from time zero to infinity, estimated using the log-linear trapezoidal method to the last collection time with extrapolation to infinity), and t_{1/2,z} (terminal half-life).

Maximum concentrations of levodopa in the blood (Cₘₐₓ values) and the area under blood concentration versus time curve (AUC) values after intracolonic dosing of a colonically absorbable form of levodopa and carbidopa were significantly higher (>2-fold) than those achieved for colonic administration of levodopa with carbidopa.

Intracolonic administration of levodopa and intraperitoneal administration of carbidopa results in very low relative bioavailability of levodopa (i.e., only 3% of intracolonically administered levodopa). By comparison, intracolonic administration of a colonically absorbable form of levodopa with intraperitoneal administration of carbidopa exhibited improved relative intracolonic bioavailability of levodopa by at least 2-fold. The data demonstrates that colonically absorbable forms of levodopa can be formulated as compositions suitable for effective sustained oral release and uptake of a colonically absorbable form of levodopa from the colon.

### Example 2

### Animal Model for Assessing Therapeutic Efficacy for Treating Schizophrenia

Morris Water Maze

The Morris Water Maze (MWM) is used as a well-validated hippocampus dependent test of visual-spatial memory. The MWM tests the ability of an animal to locate a hidden platform submerged under water by using extra-maze cues from the test environment. Rats are trained in a pool 1.8 m in diameter and 0.6 m high, containing water at about 26° C. A 10 cm square transparent platform is hidden in a constant position 1 cm below the water level in the pool. Only distal visuo-spatial cues are available to the rats for location of the submerged platform. The rats are given trials to find the hidden platform. The escape latency, i.e., the time required by the rats to find and climb onto the platform, is recorded for up to 120 s. Each rat is allowed to remain on the platform for 30 s, after which it is removed to its home cage. If the rat did not find the platform within 120 s, it is manually placed on the platform and returned to its home cage after 30 s.

Male Sprague-Dawley rats weighing 150-200 g are used. Ten days before the beginning of the experiments, the rats are handled once daily to reduce experimental stress. A composition provided by the present disclosure or control is administered to the rats for three consecutive days before behavioral testing. On each day of behavioral testing the rats are injected with either haloperidol or saline 30 min before behavioral assessment.

PCP-Induced Hyperactivity Model

Male C57Bl/6J mice from Jackson Laboratories (Bar Harbor, Me.) are used. Mice are received at 6-weeks of age. Upon receipt, mice are assigned unique identification numbers (tail marked) and are group housed with 4 mice/cage in OPTI mouse ventilated cages. All animals remain housed in groups of four during the study. All mice are acclimated to the colony room for at least two weeks prior to testing and are subsequently tested at an average age of 8 weeks of age. During the period of acclimation, mice and rats are examined on a regular basis, handled, and weighed to assure adequate health and suitability. Animals are maintained on a 12/12 light/dark cycle. The room temperature is maintained between 20° C. and 23° C. with a relative humidity maintained between 30% and 70%. Chow and water are provided ad libitum for the duration of the study. In each test, animals are randomly assigned across treatment groups. All animals are euthanized at the end of the study.

Test compounds are prepared and administered according to the following procedures. A composition provided by the present disclosure is dissolved in sterile injectable water and administered i.p. at a dose volume of 10 mL/kg at 60 min prior to PCP injection. The amount of a colonically absorbable form of levodopa administered can range, for example, from 0.01 mg/kg to 100 mg/kg. As a positive control, clozapine (1 mg/kg) is dissolved in 10% DMSO and administered i.p. at a dose volume of 10 mL/kg at 30 min prior to PCP injection. PCP (5 mg/kg) is dissolved in sterile injectable water and administered i.p. at a dose volume of 10 mL/kg.

The Open Filed (OF) test is used to assess both anxiety and locomotor behavior. The open field chambers are Plexiglas square chambers (27.3×27.3×20.3 cm; Med Associates Inc., St Albans, Vt.) surrounded by infrared photobeams (16×16×16) to measure horizontal and vertical activity. The analysis is configured to divide the open field into a center and periphery zone. Distance traveled is measured from horizontal beam breaks as a mouse moves, and rearing activity is measured from vertical beam breaks.

Mice are acclimated to the activity experimental room for at least 1 hr to prior to testing. Eight animals are tested in each run. Mice are injected with water or a composition provided by the present disclosure, placed in holding cages for 30 min, and then in the OF chamber for 30 min, removed from the OF chamber and injected with either water or PCP and returned to the OF chambers for a 60-minute session. A different group of mice are injected with either 10% DMSO or clozapine and placed in the OF chamber for 30 min, removed from the OF chamber and injected with PCP (5 mg/kg), and returned to the OF chambers for a 60-minute session.

Data is analyzed by analysis of variance (ANOVA) followed by post-hoc comparisons with Fisher Tests when appropriate. Baseline activity is measured during the first 30 min of the test prior to PCP injection. PCP-induced activity is measured during the 60 min following PCP injection. Statistical outliers that fall above or below 2 standard deviations from the mean are removed from the final analysis. An effect is considered significant if p<0.05.

Auditory Startle and Prepulse Inhibition of Startle (PPI)

Young, adult male C57Bl/6J mice from Jackson Laboratories (Bar Harbor, Me.) are used in this study. Mice are received at 6-weeks of age. Upon receipt, mice are assigned unique identification numbers (tail marked) and are group housed in standard mouse cages. All animals remain housed in groups of four during the study. All mice are acclimated to the colony room for at least two weeks prior to testing and are subsequently tested at an average age of 8-9 weeks of age. During the period of acclimation, mice are examined on a regular basis, handled, and weighed to assure adequate health and suitability. Mice are maintained on a 12 h/12 h light/dark cycle with the light on at 7:00 a.m. The room temperature is maintained between 20° C. and 23° C. with a relative humidity maintained between 30% and 70%. Feed and water are provided ad libitum during the study. For testing, animals are randomly assigned across treatment groups and balanced by PPI chamber.

Test compounds are prepared and administered according to the following procedures. A composition provided by the present disclosure is dissolved in sterile injectable water and administered i.p. at a dose volume of 10 mL/kg at 60 min prior to testing. The amount of a colonically absorbable form of levodopa and antipsychotic agent administered can range, for example, from 0.01 mg/kg to 100 mg/kg. Haloperidol (1 mg/kg) is dissolved in 10% DMSO and administered i.p. 30 minutes prior to testing the normal mouse-PPI portion of the study. As a positive control, clozapine (3 mg/kg) is dissolved in 1% Tween and administered i.p. 60 min prior to testing the PCP-PPI portion of the study. PCP (8 mg/kg) is dissolved in sterile injectable water and administered 30 minutes prior to testing. All compounds are delivered at a dose volume of 10 mL/kg.

Acoustic startle measures an unconditioned reflex response to external auditory stimulation. PPI consisting of an inhibited startle response (reduction in amplitude) to an auditory stimulation following the presentation of a weak auditory stimulus or prepulse, has been used as a tool for the assessment of deficiencies in sensory-motor gating, such as those seen in schizophrenia. Mice are placed in the PPI chamber (Med Associates) for a 5 min session of white noise (70 dB) habituation. A test session begins immediately after the 5 min acclimation period. The session starts with a habituation block of 6 presentations of the startle stimulus alone, followed by 10 PPI blocks of 6 different types of trials. Trial types are: null (no stimuli), startle (120 dB), startle plus prepulse (4, 8 and 12 dB over background noise i.e., 74, 78 or 82 dB) and prepulse alone (82 dB). Trial types are presented at random within each block. Each trial begins with a 50 ms null period during which baseline movements are recorded. There is a subsequent 20 ms period during which prepulse stimuli are presented and responses to the prepulse measured. Following a 100 ms pause, the startle stimuli are presented for 40 ms and responses are recorded for 100 ms from startle onset. Responses are sampled every ms. The inter-trial interval is variable with an average of 15 s (range from 10 to 20 s). In startle alone trials the basic auditory startle is measured and in prepulse plus startle trials the amount of inhibition of the normal startle is determined and expressed as a percentage of the basic startle response (from startle alone trials), excluding the startle response of the first habituation block.

For the normal mouse-PPI portion of the study, C57BL/6J mice are treated with vehicle, haloperidol or composition comprising a colonically absorbable form of levodopa and an antipsychotic agent and placed back in their holding cages. Thirty min following injection of vehicle or haloperidol and 60 min following injection of vehicle or a composition provided by the present disclosure, normal mouse-PPI testing commenced.

For the PCP-PPI portion of the study, C57BL/6J mice were treated with vehicle, clozapine, or a composition provided by the present disclosure and returned to their holding cages. Thirty min later, all treatment groups are injected with vehicle or PCP. Thirty min following vehicle or PCP injection, PPI testing commences.

Mice are returned to holding cages and sacrificed immediately following testing.

Data is analyzed by analysis of variance (ANOVA) followed by post-hoc analysis when appropriate. An effect is considered significant if p<0.05. For the PPI analysis, all mice that had a startle response below 100 are removed from the analysis.

Finally, it is to be noted that there are alternative ways of implementing the embodiments disclosed herein. Accordingly, the present embodiments are to be considered as illustrative and not restrictive, and the claims are not to be limited to the details given herein, but may be modified within the scope and equivalents thereof.

What is claimed is:

1. A pharmaceutical composition for oral administration comprising:
at least one antipsychotic agent in an amount that is effective for treating a positive symptom of schizophrenia in a patient; and
at least one colonically absorbable form of levodopa in an amount that is effective for treating a negative or cognitive symptom of schizophrenia in the patient, and that does not exacerbate or induce a positive symptom of schizophrenia in the patient.

2. The pharmaceutical composition of claim 1, wherein the at least one antipsychotic agent is a typical antipsychotic.

3. The pharmaceutical composition of claim 2, wherein the typical antipsychotic is chosen from chlorpromazine, haloperidol, fluphenazine, loxapine, mesoridazine, molindone, perphenazine, pimozide, raclopride, remoxipride, thioridazine, thiothixene, trifluoperazine, a pharmaceutically acceptable salt of any of the foregoing, a pharmaceutically acceptable solvate of any of the foregoing, and a combination of any of the foregoing.

4. The pharmaceutical composition of claim 1, wherein the at least one colonically absorbable form of levodopa is a levodopa prodrug.

5. The pharmaceutical composition of claim 4, wherein the levodopa prodrug provides a levodopa plasma AUC in a patient following colonic administration that is at least two times greater than the levodopa plasma AUC in the patient following colonic administration of an equivalent amount of levodopa in an equivalent dosage form.

6. The pharmaceutical composition of claim 4, wherein the levodopa prodrug is chosen from a compound of Formula (I), a compound of Formula (II), the compound of Formula (III), a compound of Formula (IV), a compound of Formula (V), a compound of Formula (VI), a pharmaceutically acceptable salt of any of the foregoing, a pharmaceutically acceptable solvate of any of the foregoing, and a combination of any of the foregoing.

7. The pharmaceutical composition of claim 6, wherein the levodopa prodrug is a compound of Formula (III) and is (2R)-2-phenylcarbonyloxypropyl (2S)-2-amino-3-(3,4-dihydroxyphenyl)propanoate mesylate.

8. The pharmaceutical composition of claim 1, further comprising an L-aromatic amino acid decarboxylase inhibitor.

9. The pharmaceutical composition of claim 1, which is a sustained release oral formulation for colonic absorption.

10. The pharmaceutical composition of claim 9, which is capable of providing a therapeutically effective concentration of the at least one antipsychotic agent and levodopa in the plasma of a patient during a continuous period of time chosen from at least about 4 hours, at least about 8 hours, at least about 16 hours, and at least about 24 hours, following oral administration of the sustained release oral formulation to the patient.

11. A method of treating schizophrenia in a patient comprising orally administering to a patient in need of such treatment:
at least one antipsychotic agent in an amount that is effective for treating a positive symptom of schizophrenia in the patient; and
at least one colonically absorbable form of levodopa in an amount that is effective for treating a negative or cognitive symptom of schizophrenia in the patient and that does not exacerbate or induce a positive symptom of schizophrenia in the patient.

12. The method of claim 11, wherein administering the at least one antipsychotic agent and the at least one colonically absorbable form of levodopa comprises administering a pharmaceutical composition comprising the at least one antipsychotic agent and the at least one colonically absorbable form of levodopa.

13. The method of claim 12, wherein the pharmaceutical composition is a sustained release oral formulation for colonic absorption.

14. The method of claim 11, wherein the at least one antipsychotic agent is a typical antipsychotic and the at least one colonically absorbable form of levodopa is a levodopa prodrug chosen from a compound of Formula (I), a compound of Formula (II), the compound of Formula (III), a compound of Formula (IV), a compound of Formula (V), a compound of Formula (VI), a pharmaceutically acceptable salt of any of the foregoing, a pharmaceutically acceptable solvate of any of the foregoing, and a combination of any of the foregoing.

15. The method of claim 11, wherein the levodopa prodrug is administered in an amount of about 100 mg-equivalents to about 1,000 mg-equivalents of levodopa per day.

16. The method of claim 11, wherein the levodopa prodrug is administered in an amount of about 200 mg-equivalents to about 800 mg-equivalents of levodopa per day.

17. The method of claim 11, which provides a plasma levodopa concentration ranging from about 50 ng/mL to about 1,000 ng/mL during a continuous period of time chosen from at least about 4 hours, at least about 8 hours, at least about 16 hours, and at least about 24 hours, following oral administration of the at least one colonically absorbable form of levodopa to the patient.

18. The method of claim 11, which provides a mean plasma levodopa concentration ranging from about 50 ng/mL to about 500 ng/mL during a continuous period of time chosen from at least about 4 hours, at least about 8 hours, at least about 16 hours, and at least about 24 hours, following oral administration of the at least one colonically absorbable form of levodopa to the patient.

19. The method of claim 11, wherein a therapeutically effective concentration of levodopa is maintained in the plasma of the patient during a continuous period of time chosen from at least about 4 hours, at least about 8 hours, at least about 16 hours, and at least about 24 hour after the at least one colonically absorbable form of levodopa is administered to the patient.

20. A method of treating schizophrenia in a patient comprising:
administering to a patient in need of such treatment at least one antipsychotic agent in an amount that is effective for treating a positive symptom of schizophrenia in the patient; and
orally administering to the patient at least one colonically absorbable form of levodopa in an amount that is effective for treating a negative or cognitive symptom of schizophrenia in the patient and that does not exacerbate or induce a positive symptom of schizophrenia in the patient.

21. The method of claim 20, wherein the at least one antipsychotic agent is a typical antipsychotic and the colonically absorbable form of levodopa is a levodopa prodrug chosen from a compound of Formula (I), a compound of Formula (II), the compound of Formula (III), a compound of Formula (IV), a compound of Formula (V), a compound of Formula (VI), a pharmaceutically acceptable salt of any of the foregoing, a pharmaceutically acceptable solvate of any of the foregoing, and a combination of any of the foregoing.
